(19) United States
(12) Patent Application Publication
(10) Pub. No.: US 2023/0310352 A1
(43) Pub. Date: Oct. 5, 2023

(54) NOVEL COMPOSITIONS AND METHODS FOR THE TREATMENT OF LUNG FIBROSIS

(71) Applicant: LAURENT PHARMACEUTICALS INC., Montreal (CA)

(72) Inventors: Radu Pislariu, Candiac (CA); IRENEJ KIANICKA, Rosemère (CA)

(21) Appl. No.: 18/022,611

(22) PCT Filed: Aug. 19, 2021
(86) PCT No.: PCT/CA2021/051148

§ 371 Date: Feb. 22, 2023

Related U.S. Application Data
(60) Provisional application No. 63/069,881, filed on Aug. 25, 2020.

(51) Int. Cl. A61K 31/167; A61K 31/4412; A61K 9/10; A61P 11/00

(52) U.S. Cl. CPC A61K 31/167; A61K 31/4412; A61K 9/10; A61P 11/00

(57) ABSTRACT

The present invention provides a method for preventing and/or slowing progression of and/or treating pulmonary fibrosis comprising administration of a therapeutically effective amount of fenretinide, fenretinide analog or pharmaceutically acceptable salt thereof.

### FIELD OF THE INVENTION

The present invention relates to compositions and methods of use of fenretinide (4-hydroxyphenyl retinamide) and its associated analogs or pharmaceutically acceptable salts thereof, as antifibrotic agents for preventing and/or slowing progression and/or treatment of pulmonary fibrosis.

### BACKGROUND OF THE INVENTION

Lung fibrosis, also referred to as pulmonary fibrosis, includes multiple serious medical conditions that occur when the lung tissue becomes damaged, scarred and stiff. In turn, the lungs lose their ability to transfer oxygen to the bloodstream, resulting in shortness of breath and vital organs being deprived of oxygen. The scarring of lung tissue can be caused by a multitude of factors, leading to many different types of pulmonary fibrosis with different etiologies, treatments, and prognoses.

Interstitial lung disease (ILD), which is synonymous with diffuse parenchymal lung disease, describes a group of lung fibrotic diseases that cause progressive fibrosis of the interstitium, the tissue and space around the air sacs of the lung. The most common form of ILD is idiopathic pulmonary fibrosis, which has a poor prognosis. Idiopathic pulmonary fibrosis (IPF) is a chronic interstitial lung disease of unknown cause, characterized by progressive fibrosis of the interstitium, in which patients experience worsening lung function resulting in pulmonary insufficiency. IPF has a poor prognosis and is associated with high rates of morbidity and mortality.

Other ILDs with similar pathological fibrotic alterations of the lung interstitium and progressive fibrosis include connective tissue disease-associated ILD (CTD-ILD) such as systemic sclerosis-ILD (SSc-ILD) and rheumatoid arthritis-ILD (RA-ILD), environmental/occupational fibrosing lung diseases like asbestosis and silicosis, pulmonary fibrosis caused by an infectious agent, drug-induced pulmonary fibrosis, idiopathic nonspecific interstitial pneumonia (iNSIP), desquamative interstitial pneumonia, respiratory bronchiolitis-ILD, cryptogenic organising pneumonia, acute interstitial pneumonia, idiopathic pleuroparenchymal fibroelastosis, idiopathic lymphocytic interstitial pneumonia, unclassifiable idiopathic interstitial pneumonias, sarcoidosis, chronic hypersensitivity pneumonitis (CHP), interstitial pneumonia with autoimmune features (IPAF), familial pulmonary fibrosis, lymphangioleio-myomatosis and histiocytosis X.

ILD is rare and mainly a disease of the middle-aged and elderly. The symptoms of ILD, chronic dyspnoea and cough, are easily confused with the symptoms of more common diseases, particularly chronic obstructive pulmonary disease and heart failure. ILD is infrequently seen in primary care and a precise diagnosis of these disorders can be challenging for physicians who rarely encounter them. (Zibrak J.D. et al., 2014, NPJ Prim Care Respir Med, 24:14054).

There is no cure for ILDs. The lung scarring that occurs in pulmonary fibrosis can't be reversed and no current procedures or medications have proved effective in removing the scarring from the lungs or stopping the progression of the disease. Some treatments may reduce symptoms temporarily or slow the disease's progression. At present, no drugs are specifically approved for the treatment of ILD, other than nintedanib and pirfenidone that are indicated for the treatment of idiopathic pulmonary fibrosis. For other progressive fibrosing ILDs, the mainstay of drug therapy is immunosuppression. However, it is postulated that once the response to lung injury in fibrosing ILDs has reached the stage at which fibrosis has become progressive and self-sustaining, immunosuppression is insufficient and targeted antifibrotic therapy would be required to slow disease progression (Cottin V. et al., 2018, Eur Respir Rev 28(151):180100).

A proportion of patients with ILDs may develop a progressive-fibrosing phenotype similar to IPF in clinical behavior and in many of the underlying pathogenetic mechanisms that drive a self-sustaining process of pulmonary fibrosis. IPF may be regarded as a model for chronic progressive-fibrosing ILDs. Other chronic fibrosing ILDs with a progressive phenotype include autoimmune ILD (e.g. rheumatoid arthritis-related ILD), hypersensitivity pneumonitis, idiopathic nonspecific interstitial pneumonias, unclassifiable interstitial pneumonias, systemic sclerosis-associated ILD (SSc-ILD), mixed connective tissue disease-associated ILD, fibrotic sarcoidosis and ILDs related to occupational exposures. (Cottin V. et al., 2018, Eur Respir Rev 27:180076). Characteristics of chronic progressive-fibrosing ILD include lung scarring and rapid disease progression, as assessed through worsening lung function tests, symptoms and/or imaging, and high mortality. Progressive lung scarring leads to breathlessness and respiratory failure. Lung function declines over time among these patients and can be debilitating and life-threatening. (Kolb, M. et al., 2019, Resp Res, 20:57). The only antifibrotic treatment approved is nintedanib, for patients with chronic fibrosing (scarring) interstitial lung diseases with a progressive phenotype.

Pulmonary fibrosis was also described as a pathologic response and complicating factor in acute lung injury (ALI) or acute respiratory distress syndrome (ARDS), especially in patients receiving mechanical ventilation. Many patients with ALI/ARDS survive the acute phase, but subsequently go on to die, often with evidence of significant pulmonary fibrosis. Severe fibrosis was demonstrated to be a frequent complication in ALI/ARDS as early as the 1990s. Lung histologic studies of patients with late ALI/ARDS suggested ongoing inflammatory injury, together with progressive fibrosis. Areas of exudation are found adjacent to advanced fibrosis, and epithelial and endothelial injury is pronounced in the late phase of ARDS. Patients with severe ALI or ARDS frequently require prolonged mechanical ventilation, and this in turn was associated with an increased severity of systemic organ failure. The acute inflammatory phase is followed by a chronic fibroproliferative phase in patients who remain intubated and mechanically ventilated. In patients who survive, pulmonary function tests often show restriction, consistent with the parenchymal fibrosis seen in lung biopsies or autopsy specimens. There is no drug specifically approved for addressing pulmonary fibrosis associated with ALI/ARDS or mechanical ventilation. (Cabrera-Benitez N.E. et al., 2014, Anesthesiology, 121(1): 189-198).

Further, pulmonary fibrosis is found in not just humans, but is also observed in canine species (Corcoran, B. M. et al.,1999, Vet Rec 144:611-6), feline (Cohn, L. A. et al., 2004, J Vet Intern Med, 18:632-641), and equine (Wong, D. M. et al., 2008, J Am Vet Med Assoc, 232:898-905).

Bronchiectasis is a chronic, progressive respiratory disorder characterised by irreversibly and abnormally dilated airways, persistent cough, excessive sputum production and recurrent pulmonary infections. Although bronchiectasis has become far more common than other respiratory conditions such as idiopathic pulmonary fibrosis (Maseli D.J. et al., 2017, Int J Clin Pract, 71:e12924), it remains a neglected and orphan disease with poor advances and with new challenges for physicians (Severiche-Bueno D. et al., 2019, Breathe; 15:286-295). In fibrotic lung, bronchiectasis occurs when small bronchial openings dilate due to traction force exerted by the surrounding fibrotic tissue. Recent data demonstrates that fibrotic honeycombing areas are covered by epithelium expressing bronchiolar markers, as a final consequence of a variety of pathogenic events starting from alveolar stem cell exhaustion, and ending in abnormal/dysplastic proliferation of bronchiolar epithelium (Piciucchi et al., 2016, BMC Pulm Med 16:87).

Fenretinide is being studied in the context of cystic fibrosis (CF) for its potential to control the exaggerated inflammatory response leading to exacerbations and lung destruction over time in this patient population. CF is a genetic disease caused by mutation of the Cystic Fibrosis Transmembrane Conductance Regulator (CFTR) gene that affects the CFTR ion channel and leads to dehydrated mucus in the lung, pancreas and other organs. The most affected is the lung, leading to formation of mucus plugs that blocks the airways and, in time, creates an environment for chronic pulmonary infection. CF is also characterized by abnormally activated inflammatory response in the lung, which overreact in the presence of pathogens and leads to irreversible lung damage. A hallmark of CF is the imbalance of key sphingolipids and essential fatty acids arachidonic acid (AA) and docosahexaenoic acid (DHA), which are believed to play an important role in the control of the inflammatory response and the defense against opportunistic bacteria. Fenretinide was shown to correct the levels of DHA and AA and sphingolipids imbalance in the lungs and plasma of Cftr.KO mice model, resulting in reduction of lung inflammation and significant decrease in the pulmonary load of Pseudomonas aeruginosa (Guilbault C. et al., 2008, Am J Respir Cell Mol Biol, 41(1):100-106). Despite its name, cystic fibrosis is not considered a type of ILD or pulmonary fibrosis per se, and no antifibrotic treatment has been proposed nor recommended for the treatment of CF which has historically consisted of CFTR modulators and mucolytics to address the thick mucus formation, antibiotics to treat the chronic pulmonary infection, and pancreatic enzymes to deal with pancreatic insufficiency.

Although antifibrotic effects of fenretinide were reported in the context of liver fibrosis by Qian J. at al., (CN101229147A) and skin fibrosis (Delany A.M. et al., 1993, Arthritis Rheum, 36(7):983-93), these findings cannot be extrapolated to lung fibrosis, which has a different pathogenesis and pathophysiology. Further, the prior art has taught away from the use of fenretinide in treating pulmonary fibrosis by reason of its disclosed role in stimulating serine-palmitoyl transferase (Yu, H. et al., 2013, J. Lipid Res. 54:189-201) which is contrary to the observed delay in radiation-induced pulmonary fibrosis arising from inhibition of serine-palmitoyl transferase (Gorshkova, I. et al., 2012, J Lipid Res, 53:1553-1568). Albeit similar appearance of fibrotic lesions, tissue fate differs substantially among organs. Liver displays by far the best regenerative capacity after acute injury or fibrotic lesion, as opposed to lung, heart and kidney. Generalized progressive pulmonary fibrosis is particularly devastating and, compared with fibrotic lesions in other organs, relatively mild fibrotic pulmonary lesions are fatal. While both idiopathic pulmonary fibrosis and systemic sclerosis-associated interstitial lung disease (SSc-ILD) are considered to follow similar pathways, anti-inflammatory treatment is the standard for SSc-ILD, whereas anti-inflammatory therapy worsens the prognosis of idiopathic pulmonary fibrosis (Zeisberg M. et al., 2013, Am J Physiol Cell Physiol, 304:C216-C225). The role of inflammation as an important component in IPF etiology is controversial and sometimes seen as an epiphenomenon of fibrosis, and more insight is needed to fully understand the role of innate and adaptive immune system in the initiation and perpetuation of IPF pathobiology (Heukels P. et al, 2019, Respir Med, 147:79-91). Therefore, it was not known nor obvious to use an inflammation-controlling agent such as fenretinide for the treatment of IPF. Owing to the complexity and variability of organ fibrosis, it is difficult to establish the relevance of involved mechanisms for different organs. For the same reason, extrapolation of findings from preclinical research conducted on a certain fibrotic organ (e.g. liver or skin) is not applicable for supporting research on the fibrosis of the lung. Even within the same organ, use of the most appropriate in vitro and in vivo models available is required (Jenkins R. G. et al., 2017, Am J Respir Cell Mol Biol, 56(5):667-679).

Furthermore, the literature citing use of fenretinide as a conjoint therapy in the context of lung fibrosis remains controversial and not proven by any supporting evidence. Fenretinide is cited in U.S. Pat. No. 8,247,370 as a conjoint therapy for treating fibrotic diseases including idiopathic pulmonary fibrosis, with fenretinide acting as an inhibitor of the VEGF pathway, specifically down-regulation of cell surface VEGF receptors. However, there is no data supporting this extrapolation, which speculates use of fenretinide as a VEGF receptor inhibitor as part of the conjoint therapy for the treatment of IPF. The inhibition of VEGF receptor as a therapeutic intervention in IPF is controversial, with Murray et al (Murray et al., 2017, JCI Insight, 2(16):e92192) teaching away from the use of fenretinide as a VEGF inhibitor, teaching that elevated VEGF is beneficial in pulmonary fibrosis. This is further exacerbated by the general toxicity observed with fenretinide with in vitro pulmonary cell models, as opposed to liver and skin fibrosis when tested at certain conditions and dosages. Further, mouse model LPS-induced acute respiratory distress syndrome (ARDS) shows significant increase of bronchoalveolar lavage fluid (BALF) VEGF following administration of inhaled fenretinide; demonstrating the prior art's error in suggesting utility of fenretinide as an adjacent therapeutic for treating pulmonary fibrotic diseases.

### SUMMARY OF THE INVENTION

The invention described herein arises from the novel and unexpected antifibrotic effect of fenretinide, fenretinide analog or pharmaceutically acceptable salt thereof on a subject with a pulmonary fibrosis. An object of the present invention is to provide for the beneficial treatment of pulmonary fibrosis in a subject by administration of fenretinide, fenretinide analog or pharmaceutically acceptable salt thereof, where the subject is human, cat, dog, or horse.

In one aspect, the present invention provides for a method of preventing and/or slowing progression of and/or treating pulmonary fibrosis in a subject in need, comprising administration to said subject of a therapeutically effective amount of fenretinide, fenretinide analog or pharmaceutically acceptable salt thereof. In one embodiment, the subject is human. In another embodiment, the subject is selected from the group comprising cat, dog and horse. In an alternative embodiment the pulmonary fibrosis is associated with one or more interstitial lung disease. In a further embodiment the one or more interstitial lung diseases are selected from chronic progressive-fibrosing interstitial lung diseases. In an alternative embodiment, the therapeutically effective amount of fenretinide, fenretinide analog or pharmaceutically acceptable salt thereof that is administered to a human subject gives rise to a plasma concentration of fenretinide or fenretinide analog of 0.1 μM to about 10 μM, preferably of 1 μM to about 3 μM, in said human subject. In an alternative embodiment, the therapeutically effective amount of fenretinide, fenretinide analog or pharmaceutically acceptable salt thereof that is administered to the human subject is about 1 mg to 1000 mg, preferably about 10 mg to 300 mg. In a further embodiment, fenretinide, fenretinide analog or pharmaceutically acceptable salt thereof is administered systemically to the human subject by oral route, or administered directly to the lungs by mouth or nasal inhalation. In a further embodiment, the fenretinide, fenretinide analog or salt thereof is present in amorphous form in a solid dispersion formulation comprising a matrix polymer.

In another aspect, the present invention provides for the use of fenretinide, fenretinide analog or pharmaceutically acceptable salt thereof in the preparation of a medicament for preventing and/or slowing progression of and/or treating pulmonary fibrosis, in a subject in need thereof.

In one embodiment, the subject is human. In another embodiment, the subject is selected from the group comprising cat, dog and horse. In an alternative embodiment the pulmonary fibrosis is associated with one or more interstitial lung disease. In a further embodiment the one or more interstitial lung diseases are selected from chronic progressive-fibrosing interstitial lung diseases. In an alternative embodiment, the therapeutically effective amount of fenretinide, fenretinide analog or pharmaceutically acceptable salt thereof that is administered to the human subject gives rise to a plasma concentration of fenretinide or fenretinide analog of 0.1 μM to about 10 μM, preferably of 1 μM to about 3 μM, in said human subject. In an alternative embodiment, the therapeutically effective amount of fenretinide, fenretinide analog or pharmaceutically acceptable salt thereof that is administered is about 1 mg to 1000 mg, preferably about 10 mg to 300 mg. In another embodiment, the medicament is capable of administration to the human subject systemically by oral route, or directly to the lungs by mouth or nasal inhalation. In a further embodiment, the fenretinide, fenretinide analog or salt thereof is present in amorphous form in a solid dispersion formulation comprising a matrix polymer.

In another aspect, the present invention provides for a composition comprising a therapeutically effective amount of fenretinide, fenretinide analog or pharmaceutically acceptable salt for use in treating pulmonary fibrosis, in a subject in need thereof. In one embodiment, the subject is human. In another embodiment, the subject is selected from the group comprising cat, dog and horse. In an alternative embodiment the pulmonary fibrosis is associated with one or more interstitial lung disease. In a further embodiment the one or more interstitial lung diseases are selected from chronic progressive-fibrosing interstitial lung diseases. In an alternative embodiment, the therapeutically effective amount of fenretinide, fenretinide analog or pharmaceutically acceptable salt thereof that is administered to the human subject gives rise to a plasma concentration of fenretinide or fenretinide analog of 0.1 μM to about 10 μM, preferably of 1 μM to about 3 μM, in said human subject. In an alternative embodiment, the therapeutically effective amount of fenretinide, fenretinide analog or pharmaceutically acceptable salt thereof that is administered is about 1 mg to 1000 mg, preferably about 10 mg to 300 mg. In another embodiment, the medicament is capable of administration to the human subject systemically by oral route, or directly to the lungs by mouth or nasal inhalation. In a further embodiment, the fenretinide, fenretinide analog or salt thereof is present in amorphous form in a solid dispersion formulation comprising a matrix polymer.

In another aspect, the present invention provides for a method of preventing and/or slowing progression of and/or treating bronchiectasis, acute lung injury associated lung fibrosis, acute respiratory distress syndrome associated lung fibrosis, or mechanical ventilation-associated lung fibrosis in said subject, comprising administration to said subject a therapeutically effective amount of fenretinide, fenretinide analog or pharmaceutically acceptable salt thereof. In one embodiment, the subject is human. In another embodiment, the subject is selected from other species comprising cat, dog and horse. In an alternative embodiment, the therapeutically effective amount of fenretinide, fenretinide analog or pharmaceutically acceptable salt thereof that is administered to a human subject gives rise to a plasma concentration of fenretinide or fenretinide analog of 0.1 μM to about 10 μM, preferably of 1 μM to about 3 μM, in said human subject. In an alternative embodiment, the therapeutically effective amount of fenretinide, fenretinide analog or pharmaceutically acceptable salt thereof that is administered to the human subject is about 1 mg to 1000 mg, preferably about 10 mg to 300 mg. In a further embodiment, fenretinide, fenretinide analog or pharmaceutically acceptable salt thereof is administered to a human subject systemically by oral route, or administered directly to the lungs by mouth or nasal inhalation. In a further embodiment, the fenretinide, fenretinide analog or salt thereof is present in amorphous form in a solid dispersion formulation comprising a matrix polymer.

In another aspect, the present invention provides for the use of fenretinide, fenretinide analog or pharmaceutically acceptable salt thereof in the preparation of a medicament for preventing and/or slowing progression of and/or treating bronchiectasis, acute lung injury associated lung fibrosis, acute respiratory distress syndrome associated lung fibrosis, or mechanical ventilation-associated lung fibrosis in said subject. In one embodiment, the subject is selected from the group comprising human, cat, dog and horse. In an alternative embodiment, the therapeutically effective amount of fenretinide, fenretinide analog or pharmaceutically acceptable salt thereof that is administered to the human subject gives rise to a plasma concentration of fenretinide or fenretinide analog of 0.1 μM to about 10 μM, preferably of 1 μM to about 3 μM, in said human subject. In an alternative embodiment, the therapeutically effective amount of fenretinide, fenretinide analog or pharmaceutically acceptable salt thereof that is administered to the human subject is about 1 mg to 1000 mg, preferably about 10 mg to 300 mg. In another embodiment, the medicament is capable of administration to the human subject systemically by oral route, or directly to the lungs by mouth or nasal inhalation. In a further embodiment, the fenretinide, fenretinide analog or salt thereof is present in amorphous form in a solid dispersion formulation comprising a matrix polymer.

In another aspect, the present invention provides for a method of preventing and/or slowing progression of and/or treating idiopathic pulmonary fibrosis, connective tissue disease-associated interstitial lung disease such as systemic sclerosis interstitial lung disease and rheumatoid arthritis interstitial lung disease, environmental/occupational fibrosing lung diseases like asbestosis and silicosis, pulmonary fibrosis caused by an infectious agent, drug-induced pulmonary fibrosis, idiopathic nonspecific interstitial pneumonia, desquamative interstitial pneumonia, respiratory bronchiolitis-ILD, cryptogenic organising pneumonia, acute interstitial pneumonia, idiopathic pleuroparenchymal fibroelastosis, idiopathic lymphocytic interstitial pneumonia, unclassifiable idiopathic interstitial pneumonias, sarcoidosis, chronic hypersensitivity pneumonitis, interstitial pneumonia with autoimmune features, familial pulmonary fibrosis, lymphangioleio-myomatosis and histiocytosis X in a human subject in need, comprising administration to said subject of a therapeutically effective amount of fenretinide, fenretinide analog or pharmaceutically acceptable salt thereof, as an antifibrotic agent. In one embodiment, the therapeutically effective amount of fenretinide, fenretinide analog or pharmaceutically acceptable salt thereof that is administered to the human subject gives rise to a plasma concentration of fenretinide or fenretinide analog of 0.1 μM to about 10 μM, preferably of 1 μM to about 3 μM, in said human subject. In an alternative embodiment, the therapeutically effective amount of fenretinide, fenretinide analog or pharmaceutically acceptable salt thereof that is administered to the human subject is about 1 mg to 1000 mg, preferably about 10 mg to 300 mg. In a further embodiment, fenretinide, fenretinide analog or pharmaceutically acceptable salt thereof is administered to the human subject systemically by oral route, or administered directly to the lungs by mouth or nasal inhalation. In a further embodiment, the fenretinide, fenretinide analog or salt thereof is present in amorphous form in a solid dispersion formulation comprising a matrix polymer.

In another aspect, the present invention provides for the use of fenretinide, fenretinide analog or pharmaceutically acceptable salt in the preparation of a medicament for preventing and/or slowing progression of and/or treating idiopathic pulmonary fibrosis, connective tissue disease-associated interstitial lung disease such as systemic sclerosis interstitial lung disease and rheumatoid arthritis interstitial lung disease, environmental/occupational fibrosing lung diseases like asbestosis and silicosis, pulmonary fibrosis caused by an infectious agent, drug-induced pulmonary fibrosis, idiopathic nonspecific interstitial pneumonia, desquamative interstitial pneumonia, respiratory bronchiolitis interstitial lung disease, cryptogenic organising pneumonia, acute interstitial pneumonia, idiopathic pleuroparenchymal fibroelastosis, idiopathic lymphocytic interstitial pneumonia, unclassifiable idiopathic interstitial pneumonias, sarcoidosis, chronic hypersensitivity pneumonitis, interstitial pneumonia with autoimmune features, familial pulmonary fibrosis, lymphangioleio-myomatosis and histiocytosis X in a human subject in need. In an alternative embodiment the pulmonary fibrosis is associated with one or more interstitial lung disease. In a further embodiment the one or more interstitial lung diseases are selected from chronic progressive-fibrosing interstitial lung diseases. In one embodiment, the therapeutically effective amount of fenretinide, fenretinide analog or pharmaceutically acceptable salt thereof that is administered to the human subject gives rise to a plasma concentration of fenretinide or fenretinide analog of 0.1 μM to about 10 μM, preferably of 1 μM to about 3 μM, in said human subject. In an alternative embodiment, the therapeutically effective amount of fenretinide, fenretinide analog or pharmaceutically acceptable salt thereof that is administered to the human subject is about 1 mg to 1000 mg, preferably about 10 mg to 300 mg. In another embodiment, the medicament is capable of administration to a human subject systemically by oral route, or directly to the lungs by mouth or nasal inhalation. In a further embodiment, the fenretinide, fenretinide analog or salt thereof is present in amorphous form in a solid dispersion formulation comprising a matrix polymer.

In another aspect the present invention provides for a method of protecting an individual from the development of pulmonary fibrosis comprising selecting an individual with acute respiratory distress syndrome or in need of mechanical ventilation and who is free of pulmonary fibrosis, administering a therapeutically effective amount of fentretinide, fenretinide analog or pharmaceutically acceptable salt thereof, and wherein the administration is performed prior to the development of pulmonary fibrosis thereby protecting the individual from development pulmonary fibrosis or reducing the extent of pulmonary fibrosis experienced by said individual.

### BRIEF DESCRIPTION OF DRAWINGS

FIG. 1 shows fenretinide dose-dependent attenuation of α-smooth muscle actin (αSMA) mRNA transcript quantification as measured by qRT-PCR after exposure to TGF-β1 in normal human lung fibroblasts, (mean±SD, n=4);

FIG. 2 shows fenretinide dose-dependent attenuation of αSMA expression as measured by ELISA detection kit after exposure to TGF-β1 in normal human lung fibroblasts, (mean±SD, n=4);

FIG. 3 shows fenretinide dose-dependent attenuation of fibrotic marker COL1A1 mRNA transcript quantification as measured by qRT-PCR after exposure to TGF-β1 in normal human lung fibroblasts (mean±SD, n=4);

FIG. 4 shows fenretinide dose-dependent attenuation of fibrotic marker Pro-Collagen 1A1 protein levels as measured by ELISA detection kit after exposure to TGF-β1 in normal human lung fibroblasts (mean±SD, n=4);

FIG. 5 shows the antifibrotic effect of fenretinide (2 μM and 4 μM) by measuring the fluorescent intensity of αSMA (A) and pro-collagen type 1 (B) fibrotic markers in TGF-β1 induced fibrosis in microtissues (mean±SD, n=8-9);

FIG. 6 shows the contractile force measurement of microtissues under TGF-β1 treatment and preventative TGF-β1+fenretinide (Fenret) treatment (2 μM and 4 μM), (mean±SD, n=6),

FIG. 7 shows the contractile force measurement of microtissues under TGF-β1 treatment and therapeutic TGF-β1+fenretinide treatment;

FIG. 8 shows immunostaining of α-SMA and pro-collagen type 1 in fibrosis-induced long microtissues (TGF-β1 treated) and under TGF-β1+Fenretinide treatment (2 μM and 4 μM);

FIG. 9 shows the ratio of the long tissue opening area/total tissue area for different treatment conditions at 3 different timepoints, (mean±SD, n=17);

FIG. 10 shows (A) normalized fluorescent intensity of α-SMA in long microtissues under TGF-β1 treatment and TGF-β1+Fenretinide treatment (2 μM and 4 μM); (B) normalized fluorescent intensity of pro-collagen in long microtissues under TGF-β1 treatment and TGF-β1+Fenretinide treatment (2 μM and 4 μM), (mean±SD, n=18);

FIG. 11 shows day 28 assessment of Ashcroft scoring of mouse lung tissue following induction of lung fibrosis by intratracheal bleomycin instillation and 21-day oral administration of LAU-7b spray dry intermediate formulation of fenretinide, (mean bar and individual values);

FIG. 12 shows day 28 assessment of fibrotic pulmonary foci by automated Sirius red staining analysis of mouse lung tissue following induction of lung fibrosis by intratracheal bleomycin instillation and 21-day oral administration of LAU-7b spray dry intermediate formulation of fenretinide (mean±SEM), *** - p<0.001 vs Sham, * - p<0.05 vs BLM Control;

FIG. 13 shows day 28 collagen assessment by whole lobe, high resolution, automated digital image analysis of mouse lung tissue stained with Sirius red following induction of lung fibrosis by bleomycin and 21-day oral administration of LAU-7b spray dry intermediate formulation of fenretinide (mean±SEM), *** - p<0.001 vs Sham, * - p<0.05 vs BLM Control, ** - p<0.01 vs BLM Control;

FIG. 14 shows day 21 assessment of Ashcroft scoring of H&E stained mouse lung tissue following induction of lung fibrosis by bleomycin instillation and 14-day oral administration of LAU-7b spray dry intermediate formulation of fenretinide (mean bar and individual values);

FIG. 15 shows day 21 assessment of fibrotic pulmonary foci by automated Sirius red staining analysis of mouse lung tissue following induction of lung fibrosis by bleomycin and 14-day oral administration of LAU-7b spray dry intermediate formulation of fenretinide (mean±SEM), *** - p<0.001 vs Sham, * - p <0.05 vs BLM Control; and

FIG. 16 shows day 21 collagen assessment by whole lobe, high resolution, automated digital image analysis of mouse lung tissue stained with Sirius red following induction of lung fibrosis by BLM instillation and 14-day oral administration of LAU-7b spray dry intermediate formulation of fenretinide (mean±SEM), *** - p<0.001 vs Sham, * - p<0.05 vs BLM Control.

### DETAILED DESCRIPTION OF THE PRESENT INVENTION

The present invention provides for novel methods and compositions useful for the treatment of pulmonary fibrosis.

As used herein, the term “about” will be understood by one skilled in the area to vary to some extent by the context under which it is used. As used herein, when referring to a measurable value such as an amount, time duration, and the like; the term “about” shall encompass variations of +/−20%, or +/−10%, more preferably +/−5%, even more preferably +/−1%, and still more preferably +/−0.1% from the specified value, as such variations are appropriate to perform the disclosed methods.

As used herein, “administration”, means providing a compound and/or composition of the present invention to a subject by any suitable method.

In another aspect of the present invention, fenretinide, fenretinide analog or pharmaceutically acceptable salt thereof may be co-administered along with another compound and/or composition that may also treat any of the diseases or disorders contemplated within the invention. In one embodiment, the co-administered compounds and/or compositions are administered separately, or in any kind of combination as part of a single therapeutic approach. The co-administered compound and/or composition may be formulated in any kind of combination as mixtures of solids and liquids under a variety of solid, gel, and liquid formulations, and as a solution.

As used herein, “alkyl”, by itself or as part of another substituent, means, unless otherwise stated, a straight or branched chain hydrocarbon having the number of carbon atoms designated and includes straight, branched chain, or cyclic substituent groups. Examples include methyl, ethyl, propyl, isopropyl, butyl, isobutyl, tert-butyls, pentyl, neopentyl, hexyl and cyclopropylmethyl.

As used herein, “slowing progression” means to ameliorate, decrease, suppress, attenuate, diminish, arrest, or stabilize the development or progression of a disease or disorder.

As used herein, an “amorphous solid dispersion” means a dispersion in which at least a major portion (i.e. more than 50%) of the fenretinide, fenretinide analog, or salt thereof in the dispersion is in amorphous form. By “amorphous” is meant that the fenretinide, fenretinide analog, or salt thereof is in a non-crystalline state. In embodiments, at least 55, 60, 65, 70, 75, 80, 85, 90% or 95% of the fenretinide, fenretinide analog, or salt thereof (by weight) in the dispersion is in the amorphous form.

As used herein, the term “composition” or “pharmaceutical composition” refers to a mixture of at least one compound useful within the invention with a pharmaceutically acceptable carrier. The pharmaceutical composition facilitates administration of the compound to a subject.

As used herein, an “effective amount” means the amount of a compound that is required to ameliorate the symptom of a disease relative to an untreated subject. The effective amount of active compound(s) used to practise the present invention for therapeutic treatment of a disease varies depending upon the manner of administration, the age, body weight, and general health of the subject. Ultimately, the attending physician or veterinarian will decide the appropriate amount and dosage regimen. Such amount is therefore referred to as an “effective amount”.

As used herein, “excipient” is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present invention is not limited in these respects. In certain embodiments, a composition of the present invention includes excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. Those skilled in the art recognize that a single excipient can fulfill more than two functions at once, e.g., can act as both a binding agent and a thickening agent and these terms are not necessarily mutually exclusive.

As used herein, “pharmaceutically acceptable” means a material, such as a carrier or diluent, which does not abrogate the biological activity or properties of the compound useful within the present invention and is relatively nontoxic. It is intended that “pharmaceutically acceptable” materials may be administered to a subject without causing undesirable biological effects or interacting in a deleterious manner with any of the components of the composition in which it is contained.

As used herein, “pharmaceutically acceptable salt” means a salt of the administered compounds prepared from pharmaceutically acceptable nontoxic acids, including inorganic acids, organic acids, inorganic bases, organic bases, solvates, hydrates, or clathrates thereof.

The compounds described herein may form salts with acids or bases, and such salts are included in the present invention. In one embodiment, the salts are pharmaceutically acceptable salt. The term “salts” includes addition of free acids or bases that are useful within the methods of the present invention. The term “pharmaceutically acceptable salt” refers to salts that possess toxicity profiles within a range that affords utility in pharmaceutical and disease and disorder treatment of patient applications. Pharmaceutically unacceptable salts may nonetheless possess properties which have utility in the practise of the present invention, and one skilled in the art would be capable of identifying and using a pharmaceutically unacceptable salt as part of the treatment of a disease or disorder of patients, as contemplated herein, or as part of the manufacturing of a compound of the present invention.

As used herein, “solid dispersion” means a solid material, in which a drug (e.g., fenretinide) is dispersed in the solid matrix polymer. Such solid dispersions are also referred to in the art as “molecular dispersions” or “solid solutions” of the drug in the polymer. Solid dispersions may be obtained by various techniques, for example fast evaporation, spray-drying, precipitation or melt extrusion (e.g., hot melt extrusion, HME). In an embodiment, the solid dispersion is obtained by spray-drying (spray-dried solid dispersion).

As used herein “Interstitial lung Disease” or “ILD” means a lung fibrotic disease that gives rise to progressive fibrosis of the tissue and space around the air sacs of the lung and includes ILDs with similar pathological fibrotic alterations in the lung interstitium and a progressive fibrosis include connective tissue disease-associated ILD (CTD-ILD) such as systemic sclerosis-ILD (SSc-ILD) and rheumatoid arthritis-ILD (RA-ILD), environmental/occupational fibrosing lung diseases like asbestosis and silicosis, pulmonary fibrosis caused by an infectious agent, drug-induced pulmonary fibrosis, idiopathic nonspecific interstitial pneumonia (iNSIP), desquamative interstitial pneumonia, respiratory bronchiolitis-ILD, cryptogenic organising pneumonia, acute interstitial pneumonia, idiopathic pleuroparenchymal fibroelastosis, idiopathic lymphocytic interstitial pneumonia, unclassifiable idiopathic interstitial pneumonias, sarcoidosis, chronic hypersensitivity pneumonitis (CHP), interstitial pneumonia with autoimmune features (IPAF), familial pulmonary fibrosis, lymphangioleio-myomatosis and histiocytosis X. It is contemplated that ILDs includes chronic progressive fibrosing ILD.

Fenretinide and Analogs Thereof

Fenretinide is a small molecule synthetic retinoid derivative and an investigational drug (new chemical entity) with well-documented history of safety in nonclinical and clinical studies. Initially explored for prevention and treatment of cancer, fenretinide was also studied for non-oncological indications such as age-related macular degeneration and cystic fibrosis. Fenretinide (4-hydroxyphenyl retinamide); also referred to as 4-HPR, with CAS registry number 65646-68-6, is a small molecule synthetic retinoid derivative and an investigational drug (new chemical entity) with well-documented history of safety in nonclinical and clinical studies. Fenretinide can be described by Formula I:

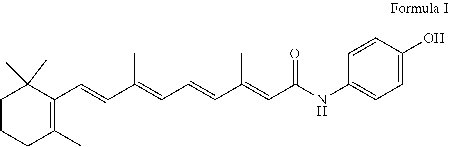 [SMILES: CC1=C(/C=C/C(C)=C/C=C/C(C)=C/C(=O)Nc2ccc(O)cc2)C(C)(C)CCC1]

Functional analogs (and/or metabolites) of fenretinide (i.e. which exhibit the same biological activity as fenretinide) may also be used in accordance with the present invention. As used herein, a “fenretinide analog” refers to a compound that shares certain chemical structural features with fenretinide but at the same time comprises one or more modifications thereto, and which exhibits similar biological activity as fenretinide (but may exhibit such activity to a different extent). Examples of analogs of fenretinide that may be used include, but are not limited to, 4-oxo-N-(4-hydroxyphenyl)retinamide (4-oxo-4-HPR), N-(4-methoxyphenyl)retinamide (4-MPR), 4-Hydroxybenzylretinone, C-glycoside and arylamide analogues of N-(4-hydroxyphenyl) retinamide-O-glucuronide, including but not limited to 4-(retinamido)phenyl-C-glucuronide, 4-(retinamido)phenyl-C-glucoside, 4-(retinamido)benzyl-C-xyloside; and retinoyl β-glucuronide analogues such as, for example, 1-(β-D-glucopyranosyl) retinamide, 1-(D-glucopyranosyluronosyl) retinamide and bexarotene, described in WO 07/136636, U.S. Patent Application No. 2006/0264514, U.S. Pat. Nos. 5,516,792, 5,663,377, 5,599,953, 5,574,177, Anding et al. (Anding, A. L. et al., 2007, Cancer Res, 67(13):6270-7) and Bhatnagar et al. (Bhatnagar, R., et al., 1991, Biochem Pharm, 41(10):1471-1477). In an embodiment, the fenretinide/fenretinide analog is represented by formula II:

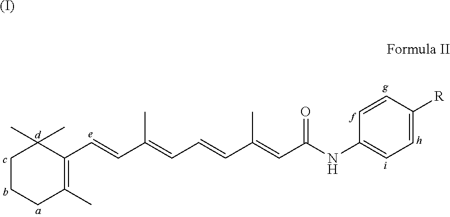

R is OH, COOH, CH₂OH, CH₂CH₂OH, or CH₂COOH; carbons a-d and f-i are optionally substituted with one or more groups selected from CH₃, OH, COOH, (CH₃)₂ and CH₂OH, or any combination thereof, and carbon e is optionally substituted with a C1-C3 alkyl group that is optionally substituted with CH₃ and/or OH.

Any pharmaceutically acceptable salts of fenretinide or fenretinide analogs may also be used in the method or use described herein.

The method or use comprises the administration or use of fenretinide or an analog of fenretinide, or a pharmaceutically acceptable salt thereof to a human subject for slowing progression or treatment of pulmonary fibrosis.

Dosage

Any suitable amount of fenretinide, fenretinide analog or salt thereof may be administered to a subject. The dosages will depend on many factors including the mode of administration. Typically, the amount of fenretinide, fenretinide analog or pharmaceutically acceptable salt thereof, contained within a single dose will be an amount that effectively prevent, slow progression or treat the pulmonary fibrosis, without inducing significant toxicity.

For prophylaxis, treatment or reduction in the severity of pulmonary fibrosis, the appropriate dosage of the compound/composition may depend on the severity of the condition, whether the compound/composition is administered for preventive or therapeutic purposes, previous or concomitant therapy, the patient's clinical history and response to the compound/composition, and the discretion of the attending physician. The fenretinide, fenretinide analog or pharmaceutically acceptable salt thereof, is/are suitably administered to the patient at one time, over a series of treatments, or as a continuous treatment.

The present invention provides dosages for the compounds and compositions comprising same. For example, depending on the severity of the disease, the effective dose may be 0.1 mg/kg, 0.5 mg/kg, 1 mg/kg, 5 mg/kg, 10 mg/kg, 15 mg/kg, 20 mg/kg/ 25 mg/kg, 30 mg/kg, 35 mg/kg, 40 mg/kg, 45 mg/kg, and up to 100 mg/kg. A typical daily dosage might range from about 1 mg/kg to 20 mg/kg or more, depending on the factors mentioned above; provided by way of administration to a patient of fenretinide, fenretinide analog or pharmaceutically acceptable salt thereof that is administered in an amount of 1 mg to about 1000 mg, preferably about 10 mg to 300 mg. For repeated administrations over several days or longer, the treatment is sustained until a desired clinical outcome occurs. The present invention contemplates establishing a plasma concentration in the patient of fenretinide or fenretinide analog of about 0.1 μM to about 10 μM, preferably of about 1 μM to about 3 μM.

However, other dosage regimens may be useful. The progress of this therapy is easily monitored by conventional techniques and assays. The disclosed dosages are intended as guidelines since the actual dose must be carefully selected and titrated by the attending physician or pharmacist based upon clinical factors unique to each patient. The optimal daily dose will be determined by methods known in the art and will be influenced by factors such as the age of the patient and other clinically relevant factors. In addition, patients may be taking medications for other diseases or conditions. The other medications may be continued during the time that fenretinide, fenretinide analog or pharmaceutically acceptable salt thereof, is given to the patient, but it is particularly advisable in such cases to begin with lower doses to determine if adverse side effects are experienced.

Compositions

The fenretinide, fenretinide analog or pharmaceutically acceptable salt thereof, may be combined with one or more optional carriers or excipients to formulate the compound(s) into suitable dosage formulations, such as tablets, capsules (e.g., hard gelatine capsules), caplets, suspensions, powders for suspensions, and the like. Such compositions may be prepared by mixing the active ingredient (e.g., fenretinide) having the desired degree of purity with one or more optional pharmaceutically acceptable carriers, excipients and/or stabilizers in a manner well known in the pharmaceutical art. Supplementary active compounds can also be incorporated into the compositions. The carrier/excipient can be suitable, for example, for oral, intravenous, parenteral, subcutaneous, intramuscular, intranasal, or pulmonary by mouth or nasal inhalation (e.g., aerosol) administration (see Remington: The Science and Practice of Pharmacy, by Loyd V Allen, Jr, 2012, 22nd edition, Pharmaceutical Press; Handbook of Pharmaceutical Excipients, by Rowe et al., 2012, 7th edition, Pharmaceutical Press). Therapeutic formulations are prepared using standard methods known in the art.

Examples of matrix materials, fillers, or diluents include, without limitation, lactose, mannitol, xylitol, microcrystalline cellulose, dibasic calcium phosphate (anhydrous and dihydrate), starch, and any combination thereof.

Examples of disintegrants include, without limitation, sodium starch glycolate, sodium alginate, carboxy methyl cellulose sodium, methyl cellulose, and croscarmellose sodium, and crosslinked forms of polyvinyl pyrrolidone such as those sold under the trade name CROSPOVIDONE® (available from BASF Corporation), and any combination thereof.

Examples of binders include, without limitation, methyl cellulose, microcrystalline cellulose, starch, and gums such as guar gum, tragacanth, and any combination thereof.

Examples of lubricants include, without limitation, magnesium stearate, calcium stearate, stearic acid, and any combination thereof.

Examples of glidants include, without limitation, metal silicates, silicon dioxides, higher fatty acid metal salts, metal oxides, alkaline earth metal salts, and metal hydroxides. Examples of preservatives include, without limitation, sulfites (an antioxidant), benzalkonium chloride, methyl paraben, propyl paraben, benzyl alcohol, sodium benzoate, and any combination thereof.

Examples of suspending agents or thickeners, without limitation, include xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, titanium dioxide, and any combination thereof.

Examples of anticaking agents or fillers, without limitation, include silicon oxide, lactose, and any combination thereof.

Examples of solubilizers include, without limitation, ethanol, propylene glycol, polyethylene glycol, and any combination thereof.

Examples of antioxidants include, without limitation, phenolic-based antioxidants such as butylated hydroxyanisole (BHA), butylated hydroxytoluene (BHT), tert-butyl-hydroquinone (TBHQ), 4-hydroxymethyl-2,6-di-tert-butylphenol (HMBP), 2,4,5-trihydroxy-butyrophenone (THBP), propyl gallate (PG), triamyl gallate, gallic acid (GA), α tocopherol (vitamin E), tocopherol acetate, reducing agents such as L-ascorbic acid (vitamin C), L-ascorbyl palmitate, L-ascorbyl stearate, thioglycolic acid (TGA), ascorbyl palmitate (ASP), sulphite-based antioxidants such as sodium sulphite, sodium metabisulphite, sodium bisulphite and thioglycerol and other agents such as disodium ethylenediamine tetraacetate (EDTA), sodium pyrophosphate, sodium metaphosphate, methionine, erythorbic acid and lecithin, and any combination thereof. In an embodiment, the formulation comprises a combination of antioxidants. In an embodiment, the formulation comprises a combination of BHA and BHT. In an embodiment, the formulation comprises ascorbic acid.

Another class of excipients is surfactants, optionally present from about 0 to about 10 wt %. Suitable surfactants include, without limitation, fatty acid and alkyl sulfonates; commercial surfactants such as benzalkonium chloride (HYAMINE® 1622, available from Lonza, Inc., Fairlawn, N.J.); dioctyl sodium sulfosuccinate (Docusate Sodium, available from Mallinckrodt Spec. Chem., St. Louis, Mo.); polyoxyethylene sorbitan fatty acid esters (TWEEN®, available from ICI Americas Inc., Wilmington, Del.; LIPOSORB® O-20, available from Lipochem Inc., Patterson N.J.; CAPMUL® POE-0, available from Abitec Corp., Janesville, Wis.); and natural surfactants such as sodium taurocholic acid, 1-palmitoyl-2-oleoyl-sn-glycero-3-phosphocholine, lecithin, and other phospholipids and mono- and diglycerides, and any combination thereof. Such materials can be employed to increase the rate of dissolution by, for example, facilitating wetting, or otherwise increase the rate of drug release from the dosage form.

Other conventional excipients, including pigments, lubricants, flavorants, humectants, solution retarding agents, absorption accelerators, wetting agents, absorbents, and other ones well-known in the art, may be employed in the compositions of this invention. For example, excipients such as pigments, lubricants, flavorants, and so forth may be used for customary purposes and in typical amounts without adversely affecting the properties of the compositions.

Other components commonly added to pharmaceutical compositions include, e.g., inorganic salts such as sodium chloride, potassium chloride, calcium chloride, sodium phosphate, potassium phosphate, sodium bicarbonate; and organic salts such as sodium citrate, potassium citrate, sodium acetate, etc.

In an embodiment, the fenretinide, fenretinide analog or pharmaceutically acceptable salt thereof is present in the composition as an amorphous solid dispersion as described in U.S. Patent Publication No. 2017/0189356 A1, which is incorporated by reference in its entirety.

Examples of “matrix polymers”, also referred to in the field as “concentration-enhancing polymers” or “dispersion polymers”, which may be suitable for use in the present invention, are discussed in detail in for example U.S. Pat. Nos. 7,780,988 and 7,887,840. The matrix polymer can be any pharmaceutically acceptable polymer that, once co-processed with the fenretinide, fenretinide analog, or salt thereof, functions to maintain the fenretinide/ fenretinide analog in amorphous form.

Examples of polymers that may be suitable for use with the present invention comprise non-ionizable (neutral) non-cellulosic polymers. Exemplary polymers include: vinyl polymers and copolymers having at least one substituent selected from hydroxyl, alkyl acyloxy, and cyclic amido; polyvinyl alcohols that have at least a portion of their repeat units in the unhydrolyzed (vinyl acetate) form; polyvinyl alcohol polyvinyl acetate copolymers; polyvinyl pyrrolidone; and polyethylene polyvinyl alcohol copolymers; and polyoxyethylene-polyoxypropylene copolymers.

Other examples of polymers that may be suitable for use with the present invention comprise ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGIT® series, amine-functionalized polyacrylates and polymethacrylates; proteins such as gelatin and albumin; and carboxylic acid functionalized starches such as starch glycolate.

Other examples polymers that may be suitable for use with the present invention comprise nonionizable cellulosic polymers that may be used as the polymer include: hydroxypropyl methyl cellulose acetate, hydroxypropyl methyl cellulose (HPMC), hydroxypropyl cellulose, methyl cellulose, hydroxyethyl methyl cellulose, hydroxyethyl cellulose acetate, hydroxyethyl ethyl cellulose, and the like.

While specific polymers have been discussed as being suitable for use in the dispersions formable by the present invention, blends of such polymers may also be suitable. Thus, the term “matrix polymer” is intended to include blends of polymers in addition to a single species of polymer.

In an embodiment, the matrix polymer comprises polyvinylpyrrolidone. In another embodiment, the matrix polymer is a polyvinylpyrrolidone, for example polymers sold under the trade-name PLASDONE® (povidones), polyvinylpyrrolidone K12, polyvinylpyrrolidone K17, polyvinylpyrrolidone K25, polyvinylpyrrolidone K30 or polyvinylpyrrolidone K90.

In an embodiment, the ratio of the fenretinide, fenretinide analog, or salt thereof/matrix polymer is from about 1:5 to about 5:1, in further embodiments about 1:4 to about 4:1, about 1:3 to about 3:1, about 1:2 to about 2:1 or about 1.5:1 to about 1:1.5, by weight. In an embodiment, the solid dispersion comprises between about 30 to about 50% of the fenretinide, fenretinide analog, or pharmaceutically acceptable salt thereof, and between about 50 to about 70% of matrix polymer. In another embodiment, the solid dispersion comprises between about 40% of the fenretinide, fenretinide analog, or salt thereof, and about 60% of matrix polymer, by weight.

In an embodiment, the solid dispersion comprises one or more additives. Additives that may be suitable for use with the present invention comprise antioxidant agents. Exemplary antioxidants include: L-ascorbic acid (vitamin C), propyl gallate, sodium sulfite, sodium metabisulfite, sodium bisulfite, thioglycerol, thioglycolic acid, tocopherols and tocotrienols, butylated hydroxyanisole (BHA), butylated hydroxytoluene (BHT) or any combination thereof. In an embodiment, the matrix polymer or solid dispersion comprises BHA and/or BHT as antioxidant agent(s). In an embodiment, the matrix polymer or solid dispersion comprises BHA and BHT as antioxidant agents. In an embodiment, the matrix polymer comprises L-ascorbic acid as antioxidant agent. In an embodiment, the antioxidant agent(s) is/are present in an amount of about 0.01% to about 5%, in further embodiments in an amount of about 0.1% to about 5%, about 0.2% to about 4%, 0.5% to about 3% or 0.5% to about 2%.

The amorphous solid dispersion of fenretinide, fenretinide analog, or pharmaceutically acceptable salt thereof may be combined with one or more optional excipients as described above.

In an embodiment, the amorphous solid dispersion of fenretinide, fenretinide analog, or salt thereof is combined with a disintegrant, for example a cross-linked sodium carboxymethylcellulose e.g., croscarmellose (SOLUTAB®). Other examples of disintegrants include corn starch, potato starch, sodium carboxymethylcellulose, sodium starch glycolate, sodium croscarmellose, crospovidone, and any combination thereof. In an embodiment, the disintegrant is present in an amount from about 2% to about 10% by weight, for example from about 3% to about 8% or about 4% to about 6% by weight.

In an embodiment, the amorphous solid dispersion of fenretinide, fenretinide analog, or salt thereof is combined with a lubricant, for example magnesium stearate. Other examples of lubricants include talc, silicon dioxide, stearic acid, and sodium stearyl fumarate. In an embodiment, the lubricant is present in an amount from about 0.5 to about 2% by weight, for example from about 0.8 to about 1.2% or about 1% by weight.

In an embodiment, the amorphous solid dispersion of fenretinide, fenretinide analog, or 30 salt thereof is combined with a filler or diluent, for example microcrystalline cellulose (AVICEL®, such as AVICEL®PH-102) and/or calcium hydrogen phosphate dehydrate (EMCOMPRESS®). Other examples of fillers or diluents include crystalline cellulose, cellulose derivatives, acacia, corn starch, lactose, mannitol, sugars, calcium phosphate, calcium carbonate, gelatins, and any combination thereof. In an embodiment, the filler or diluent is present in an amount from about 20% to about 45% by weight, for example from about 30% to about 40% by weight, e.g., about 35%.

In an embodiment, the amorphous solid dispersion of fenretinide, fenretinide analog, or salt thereof is combined one or more antioxidants, for example butylated hydroxyanisole (BHA), butylated hydroxytoluene (BHT), citric acid, sodium metabisulfite, alpha-tocopherol and/or L-ascorbic acid.

In certain embodiments, the amorphous solid dispersion as disclosed herein is formulated as an oral dosage formulation. Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an elixir or syrup, or as pastilles (using an inert matrix, such as gelatin and glycerin, or sucrose and acacia), and the like, each containing a predetermined amount of an active ingredient. A composition may also be administered as a bolus, electuary, or paste.

In an embodiment, the oral dosage formulation is a tablet. A tablet may be made by compression or molding, optionally with one or more accessory ingredients. Compressed tablets may be prepared using binder, lubricant, inert diluent, preservative, disintegrant, surface-active or dispersing agent. Molded tablets may be made by molding in a suitable machine a mixture of the powdered inhibitor(s) moistened with an inert liquid diluent.

In some embodiments of the oral dosage formulation as disclosed herein, the amorphous solid dispersion is present in an amount of from about to about 90%, about 20 to about 80%, about 30 to about 60% or about 45 to about 55% by weight, or another range within the values provided herein.

Pulmonary Fibrosis Cellular Mechanisms and Preclinical Models

The term “pulmonary fibrosis” or “lung fibrosis” means the formation or development of excess fibrous connective tissue (fibrosis) in the lung thereby resulting in the development of scarred (fibrotic) tissue. More precisely, pulmonary fibrosis is a disease that causes swelling and scarring of the alveoli and interstitial tissues of the lungs. This damage to the lung tissue causes stiffness of the lungs which subsequently makes breathing (gas exchange) more and more difficult. Pulmonary fibrosis typically results from chronic inflammation, which is defined as an immune response that persists for several months and in which inflammation, tissue remodelling and repair processes occur simultaneously. However, there is evidence of pulmonary fibrosis resulting over shorter periods of time (weeks) during acute inflammation of the lungs resulted from conditions such as ARDS, as a result of mechanical ventilation, or both. When ARDS and/or mechanical ventilation is associated with formation of the fibrosis of the lung, the prognosis is poor and results in sharp increase in mortality in these patients. Sometimes the fibrotic process may also result in permanent abnormal dilation and destruction of the major bronchi and bronchiole walls, leading to bronchiectasis.

Fibroblasts are derived from primitive mesenchyme, are ubiquitous throughout the body, and are the primary effector cells in a variety of fibrotic conditions. A primary function of the fibroblast is to maintain tissue homeostasis through regulation of the extracellular matrix (ECM) via production and maintenance of its components. Additionally, fibroblasts provide a supportive function and have varying roles in angiogenesis, inflammation, wound healing, and restoration of tissue integrity. (Garret et al., 2017, J Scleroderma Relat Disord, 2(2):69-134; Wynn T. A. 2011, J Exp Med, 208(7):1339-1350). Fibroblasts differ from the anatomical site, the disease status, and even within the same tissue. Consistent with the varying biophysical requirements of different tissues, fibroblasts from distinct tissues differ in proliferation, collagen and matrix metalloproteinase (MMP) production, contractility, and immunomodulatory function. Importantly, these differences in characteristic phenotypes among fibroblasts from distinct tissues are maintained after extended in vitro culture, supporting the concept that fibroblasts possess positional identity. The diversity of fibroblasts can be explained by their distinct cellular origins. Fibroblasts mainly originate from primary mesenchymal cells (e.g. dermal fibroblast), but can also arise through epithelial-mesenchymal transition (as seen in the liver and kidney), or endothelial-mesenchymal transition (as seen in the lung, heart, and cancer), and can also be derived from circulating cells, including mesenchymal stromal cells. (Van Linthout S. et al, 2014, Cardiovas Res, 102:258-269)

Lung fibroblasts have an increased activity with respect to proliferation, migration, ECM synthesis and response to fibrogenic cytokines. The increased deposition of ECM from activated highly contractile and collagen-producing fibroblasts contributes to the stiffening of the lung tissue and the destruction of alveolar oxygen exchange area which results in progressive dyspnea and eventually death. Evidence favours alveolar epithelial cell injury as the initial event which triggers the release of TGF-β, a central profibrotic growth factor driving lung parenchymal fibrosis. Working in concert with additional growth factors, TGF-β1 drives the formation of fibroblast foci and the exaggerated deposition of extracellular matrix (ECM). Repeated injury at different sites within the lung generates multifocal areas of pathology at different stages of development. (Roach K. M. et al, 2018, Nature Sci Rep, 8:342)

Significantly increased lung tissue mechanical properties such as contractility is a major clinical indicator of pulmonary fibrosis. Reducing or halting the increase in lung tissue mechanical property, clinically measured as forced vital capacity (FVC), is used as an endpoint to evaluate the efficacy of antifibrosis drugs in clinical trials. Preclinical models that can recapitulate the sophisticated biomechanical condition of lung tissue and provide early efficacy of the drugs are therefore very important in correctly assessing the antifibrotic potential of a drug specifically the lung fibrosis. Human lung microtissues are a new tool that models key biomechanical events occurred during lung fibrogenesis, including progressive stiffening and contraction of alveolar tissue, and traction force-induced bronchial dilation. (Asmani M. et al, 2018, Nature Corn, 9:2066).

Recognizing the challenge in crafting a robust package of preclinical testing that replicates the particularities of lung fibrosis, the American Thoracic Society issued a series of recommendations for preclinical assessment of antifibrotic compounds. The consensus view is that the intratracheal bleomycin animal model is the best-characterized model available for preclinical testing. The bleomycin model is characterized by periods of acute lung injury (Days 0-7), fibroproliferation (Days 3-14), and established fibrosis (Days 14-28) that generally resolves over a variable time period. The consensus is that the investigational product should be administered after the acute inflammatory response has subsided, depending on the model kinetics, at least 7-days after bleomycin instillation. The study should measure the collagen accumulation along with histologic assessments as primary endpoints. Hematoxylin and eosin (H&E) and Sirius red staining (or Masson's trichrome) is used to assess fibrosis histologically, and, usually, the severity of lung fibrosis is semi-quantified in H&E stained tissue sections through an Ashcroft scoring system, where the grade of fibrosis is scored from 0 (normal lung) to 8 (total fibrous obliteration of fields) by examining randomly chosen sections. More recently, digital scoring systems using automated image analysis have been developed. (Jenkins R. G. et al, 2017, Am J Respir Cell Mol Biol, 56(5):667-679)

Observer-independent quantification of pulmonary fibrosis by using automated software histological image analysis of lung sections from mice treated with bleomycin represents a fully accurate and reliable quantification of bleomycin-induced fibrotic lung alterations. The sensitivity to quantify precisely the fibrotic lung alterations at low bleomycin concentrations is a major improvement over the traditional methods, with a high correlation between the automated analysis and standard evaluation methods like Ashcroft scoring (p<0.0001) or CT scan. This correlation establishes automated analysis as a novel end-point measure of BLM-induced lung fibrosis in mice, which will be very valuable for future preclinical drug explorations. (Gilhodes J-C et al, 2017, PLoS 12(1): e0170561).

The present invention arises from the novel and unexpected demonstration of the antifibrotic effect on multiple in vitro and in vivo models highly correlative to human lung fibrosis; with daily oral treatment with a fenretinide formulation, LAU-7b SDI, reducing, in a dose related manner, lung fibrosis developing after the intratracheal instillation of bleomycin. Further, the inclusion of higher protein content in the in vitro models, by way of fetal bovine serum inclusion in the disclosed pulmonary cellular model, reduced the toxicity of fenretinide known in the art; allowing the novel identification of the utility of fenretinide as a therapeutic for pulmonary fibrosis. Though an exact understanding of the mechanism is not necessary to practise the present invention, it is hypothesized that in the presence of high protein concentration arising from addition of fetal bovine serum to in vitro models or as naturally exists in humans or animals, the proteins bind to the fenretinide substantially reducing its free plasma concentration responsible for therapeutic activity and decreasing the toxicity, thus allowing the novel observation of its benefit in the context of pulmonary fibrosis. The following provide examples of such effects, and should not be construed as further limiting.

### Example 1: Fenretinide Antifibrotic Effects on Normal Human Lung Fibroblasts (NHLF) Treated with TGF-β

With TGF-β being one of the major activators of the transition of fibroblasts to myofibroblasts, many treatments aim to inhibit TGF-β-induced signalling pathways and their effect of specific markers of fibrosis, such as a-smooth muscle actin (α-SMA) protein and Pro-collagen 1A1 synthesis. The following demonstrates for the first time in normal human lung fibroblasts (NHLF), the ability for fenretinide to attenuate and, at certain doses, completely abolish the TGF-β1-mediated induction of α-SMA protein and Pro-collagen 1A1 markers of fibrosis, in a dose-dependent manner.

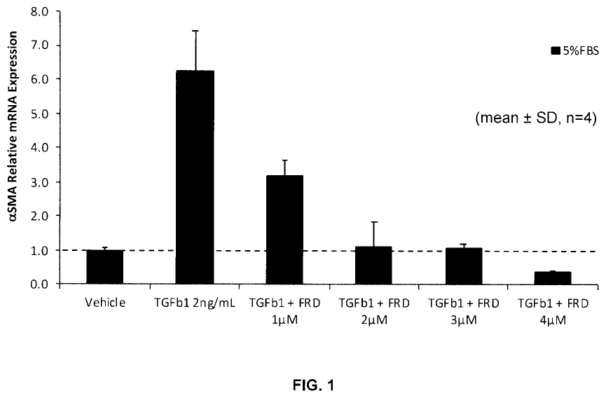

Normal human lung fibroblast cells (NHLF cells, ATCC CRL-7000, EMEM media; 5% FBS) were incubated for 3 days at 37° C. and treated with TGF-β1 (2 ng/mL) or TGF-β1 (2 ng/mL)+Fenretinide (1, 2, 3, 4 μM). At the end of the 3 day-treatment period, media was removed using a vacuum pump and total ribonucleic acid (RNA) was isolated using the TriReagent protocol (BioShop) and cDNA was obtained by reverse transcription using the SUPERSCRIPT® VILO®. αSMA transcript levels were determined by quantitative real-time Polymerase Chain Reaction (qRT-PCR) using the Fast SyBR Master Mix and specific probes, and quantified following the ΔΔCT, normalized to PUM-1 expression as internal control. The experiment was performed in quadruplicate (4 wells/condition). FIG. 1 demonstrates that fenretinide treatment attenuates, in a dose-dependent manner the induction of α-SMA fibrotic marker expression measured by qRT-PCR, after exposure to TGF-β1 (2 ng/mL) in NHLF cells.

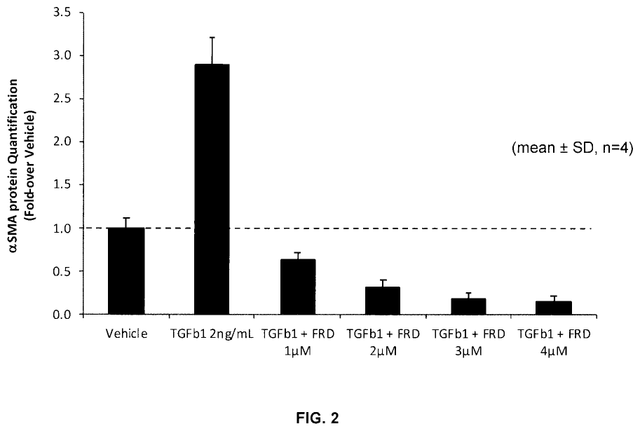

To further demonstrate the effect of fenretinide on pulmonary tissue fibrosis, α-SMA protein expression was examined using an enzyme linked immunosorbent assay (ELISA). NHLF cells were incubated for 3 days at 37° C. and treated with TGF-β1 (2 ng/mL) or TGF-β1 (2 ng/mL)+fenretinide (1, 2, 3, 4 μM). At the end of the 3 day-treatment period, media was collected in identified tubes, centrifuged at 2,000×g for 20 minutes and stored at −80° C. Cells were collected using trypsin, transferred in identified tubes and centrifuged at 1,000×g for 5 minutes. Cells were then washed twice in phosphate buffered saline (PBS) 1X prior to be resuspended in ELISA lysis buffer. α-SMA protein contents were determined using Abcam ELISA detection kit (Ab240678). The experiment was performed in quadruplicate. FIG. 2 shows that fenretinide treatment attenuates in a dose-dependent manner the induction of fibrotic marker α-SMA levels quantified using ELISA detection kit.

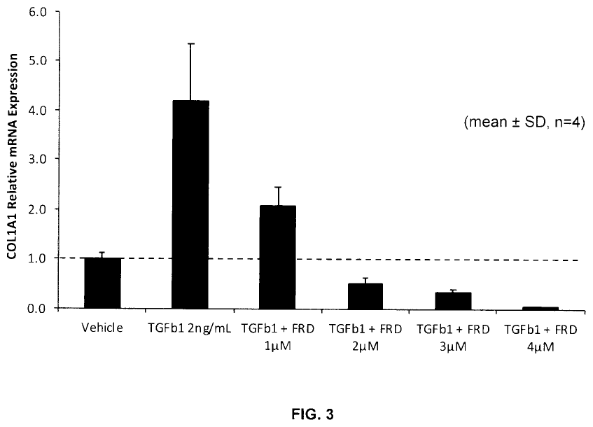

To further demonstrate the effect of fenretinide on pulmonary tissue fibrosis, induction of fibrotic marker COL1A1 gene expression (the gene encoding for Pro-collagen 1A1 protein) measured by qRT-PCR, was observed. NHLF cells were incubated for 3 days at 37° C. and treated with TGF-β1 (2 ng/mL) or TGF-β1 (2 ng/mL)+Fenretinide (1, 2, 3, 4 μM). At the end of the 3 day-treatment period, media was removed using a vacuum pump and total RNA was isolated using the TriReagent protocol (BioShop) and cDNA was obtained by reverse transcription using the SUPERSCRIPT® VILO®. COL1A1 transcript levels were determined by qRT-PCR using the Fast SyBR Master Mix and specific probes, and quantified following the ΔΔCT, normalized to PUM-1 expression as internal control. The experiments were performed in quadruplicate. FIG. 3 shows that fenretinide treatment attenuates, in a dose-dependent manner, the induction of fibrotic marker COL1A1 gene expression

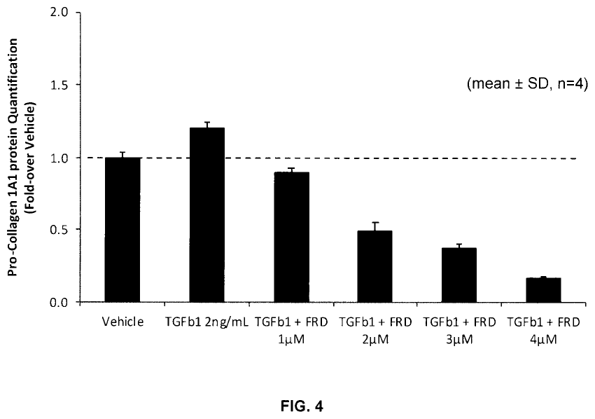

To further demonstrate the effect of fenretinide on pulmonary tissue fibrosis induction of fibrotic marker pro-collagen 1A1 protein levels quantified using ELISA detection kit, was observed. NHLF cells were incubated for 3 days at 37° C. and treated with TGF-β1 (2 ng/mL) or TGF-β1 (2 ng/mL)+Fenretinide (1, 2, 3, 4 μM). At the end of the 3 day-treatment period, media was collected in identified tubes, centrifuged at 2,000 g for 20 minutes and stored at −80° C. Cells were collected using trypsin, transferred in identified tubes and centrifuged at 1,000 g for 5 minutes. Cells were then washed twice in PBS 1X prior to be resuspended in ELISA lysis buffer. Pro-collagen 1A1 protein contents were determined using Abcam ELISA detection kit (Ab210966). Experiment performed in quadruplicate. FIG. 4 shows dose-dependent attenuation of the induction of the Pro-collagen 1A1 fibrotic marker.

### Example 2: Fenretinide Antifibrotic Effects on 3D Fibrotic Lung Microtissue Arrays Model of Lung Fibrosis

To confirm the findings of the NHLF cells, the effects of fenretinide administration was observed on a newly developed in vitro model arrays of 3D microtissues that are suspended over multiple flexible micropillars. Progression of lung fibrosis is characterized by stiffening of the membranous tissue that makes up the alveolar air sacs, caused by the accumulation of highly contractile and collagen-producing myofibroblasts. To model the physio-pathological characteristics of lung fibrosis in vitro, different experimental systems have been developed. Planar 2D culture-based assays offer multiparameter readout of fibrotic cellular markers, but they have very limited capacity to measure or reproduce tissue mechanics. In order to recapitulate dynamic changes in tissue mechanics during fibrogenesis, a newly validated membranous human lung microtissues array was used, that models key biomechanical events occurred during lung fibrogenesis, including progressive stiffening and contraction of alveolar tissue, decline in alveolar tissue compliance, and traction force-induced bronchial dilation. This new 3D array of membranous lung microtissues model of lung fibrosis had been validated using two FDA-approved antifibrosis drugs, pirfenidone and nintedanib. This model was further extended to larger lung tissue arrays, to mimic the traction on alveoli as a model of bronchiectasis. (Asmani M. et al, 2018, Nature Com, 9:2066)

The lung microtissue chip system used is based on bio-MEMS technology that can assemble 3D cell-populated submillimeter microtissues in arrays of polydimethylsiloxane (PDMS) microwells. In each microwell, human lung fibroblasts or epithelial cells spontaneously contract and assemble the matrix proteins, such as collagen, into microtissues that anchor on top of an array of micropillars. The flexible micropillars serve as in situ force sensors that can report the contractile force generated by the tissue.

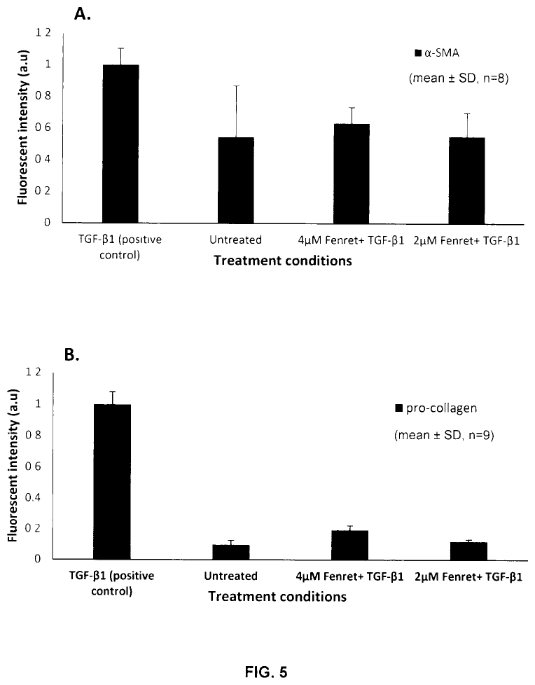

The effect of fenretinide on the inhibition of fibrotic biomarkers expression as a preventative treatment regimen, using immunostaining of fibrotic markers in fibrosis-induced microtissues was observed. shows the antifibrotic effect of fenretinide (2 μM and 4 μM) by measuring the normalized fluorescent intensity of α-SMA (A) and pro-collagen type 1 (B) fibrotic markers in TGF-β1 induced fibrosis in microtissues (mean±SD, n=8-9). Two fenretinide doses (2 μM and 4 μM) were co-administered with TGF-β1 (5 ng/ml) at the beginning of microtissue culture, and remained for 6 days. FIG. 5A shows quantified fluorescent intensity of α-SMA in microtissues under TGF-β1 (5 ng/ml) treatment and TGF-β1 (5 ng/ml)+fenretinide treatment (2 μM and 4 μM); examined at the end of day 6, as measured by quantified fluorescent intensity using a Nikon Ti-U microscope equipped with a Hamamatsu ORCA-ER CCD camera. FIG. 5B shows quantified fluorescent intensity of pro-collagen under TGF-β1 treatment and TGF-β1+fenretinide treatment. The results show that expressions of both fibrotic markers were increasingly inhibited by increasing fenretinide concentrations from 2 μM and 4 μM.

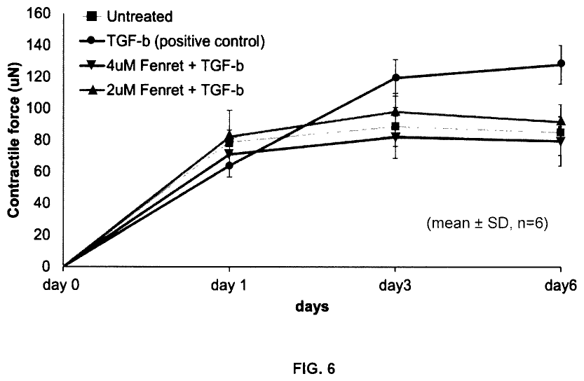

The effect of fenretinide on the inhibition of microtissue contractile force in a preventative treatment regimen was also measured. Increased contractile force is one of the main biomechanical features associated with fibrosis progression. The spontaneous contraction force generated by individual microtissues was determined from the deflection of micropillars based on cantilever bending theory. Micropillar deflection was measured using phase contrast microscopy, by comparing the deflected position of the centroid of each pillar top to the centroid of its base. Microtissue contraction force was measured for the same batch of samples on a daily basis for each pharmacological condition. The micropillar spring constant was determined based on the actual geometry of the micropillar and the elastic modulus of the PDMS. Time-lapsed force measurement showed that microtissue contractile force continuously increased under TGF-β1 (5 ng/ml) treatment to around 130 μN over a 6-day culture period. In contrast, the force developed by untreated samples plateaued after 1 day at around 80 μN. Fenretinide was able to inhibit TGF-131-induced increase in contractile force in a dose-dependent manner. FIG. 6 shows that at the 6-day measurement, the 2 μM fenretinide dose demonstrated reduction of contractile force to around 80-85 μN, a reduction of about 28% as compared to TGF-β1 only treated condition (positive control). The 4 μM fenretinide dose reduced the contractile force by Day 6 to around 70 μN, a reduction of about 38% as compared to TGF-β1 treated condition. Interestingly, the contractile force measured from Day 1 to Day 6 is approaching the levels of the samples not treated with TGF-β1 for the fenretinide 2 μM dose; consistent with an unexpected antifibrotic effect of fenretinide on human pulmonary tissues.

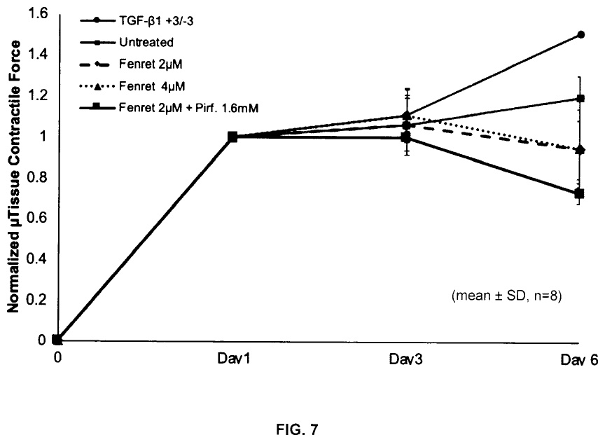

The effect of fenretinide 4 μM dose on the inhibition of microtissue contractile force was tested in a therapeutic treatment regimen. Fibrosis was first allowed to progress for three days under the induction of TGF-β1 (5 ng/ml) and fenretinide was then added from day 3 to day 6 to model a therapeutic treatment. FIG. 7 shows that removing TGF-β1 during the last 3 days (TGF-β1 +3/−3 group) did not cause an inhibition of the tissue contractility under TGF-β1 treatment, therapeutic TGF-81+fenretinide treatment (2 μM and 4 μM), and TGF-81+fenretinide (Fenret) treatment (2 μM)+pirfenidone (Pirf, 1.6 mM). TGF-β1 present in media for 3 days and absent for following 3 days (mean±SD, n=8). However, on day 6 as compared to day 3, fenretinide at 2 μM reduced the tissue contractility by 10%, 4 μM fenretinide by 15% and the combination of 2 μM fenretinide and 1.6 mM pirfenidone by 26%. Compared to the TGF-β1+3/−3 group on day 6, fenretinide, 2 μM or 4 μM reduced the tissue contractile force by 34% and the combination treatment of 2 μM fenretinide and 1.6 mM pirfenidone by 54% shows that a fenretinide dose of 4 μM was able to inhibit the contractile force by 57% as compared to TGF-β1 only treated condition (positive control).

These observations are further described in Table 1, which presents fenretinide induced reduction of alpha smooth muscle actin (α-SMA) and pro-collagen expression and contractile forces induced by TGF-β1 in lung microtissue in preventative and therapeutic treatment measured as a percentage. Table 1A, shows the results of the preventive treatment, with fenretinide (Fenret) co-administered with TGF-β1 at the beginning of lung microtissue culture and continued for 6 days; and Table 1B presenting the therapeutic effects when fibrosis was allowed to progress for 3 days under the induction of TGF-β1, with fenretinide added from day 3 to day 6 either alone or in combination with pirfenidone (Pirf), on the expression of markers of fibrosis and contractile forces.

TABLE 1
Fenretinide induced reduction of α-SMA and pro-collagen expression
and contractile forces induced by TGF-β1 in lung microtissue
in preventative and therapeutic treatment (percentage)
A. Preventative treatment - percentage change

| Fibrotic Markers | 4 μM Fenret | 2 μM Fenret |
|---|---|---|
| α-SMA | 37.0% | 45.0% |
| pro-collagen | 81.0% | 88.0% |
| contractile force | 38.0% | 28.0% |

B. Therapeutic treatment - percentage change

| | | | 2 μM Fenret + |
|---|---|---|---|
| Fibrotic Markers | 4 μM Fenret | 2 μM Fenret | 1.6 mM Pirf |
| α-SMA | 72.0% | 56.0% | 76.0% |
| pro-collagen | 58.0% | 39.0% | 68.0% |
| contractile force | 34.0% | 34.0% | 54.0% |

### Example 3: Fenretinide Antifibrotic Effects on Lung Fibroblast-Populated Long Microtissue, as a Model of Traction Bronchiectasis

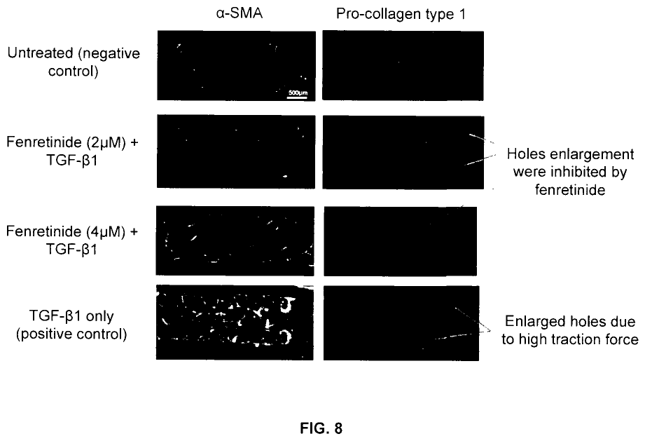

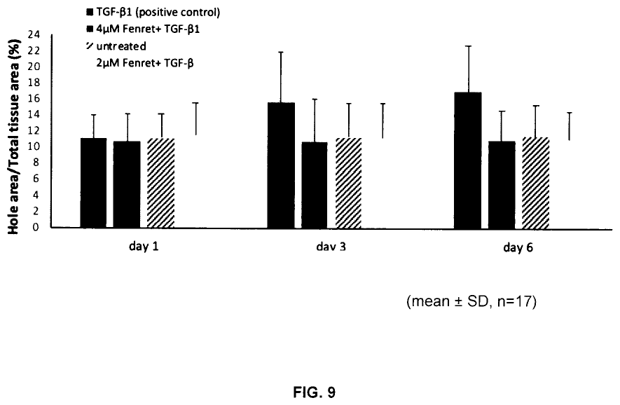

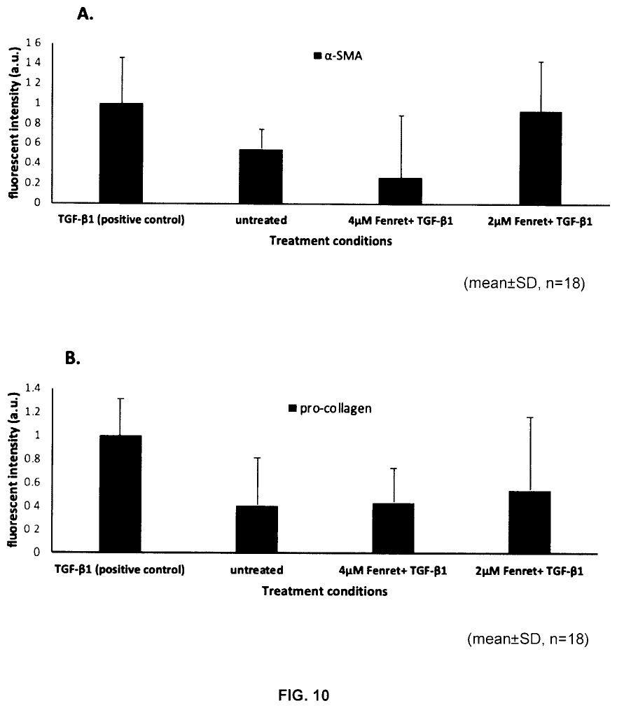

In a fibrotic lung, traction bronchiectasis occurs when small bronchial openings dilate due to traction force exerted by the surrounding fibrotic tissue. To model this unique fibrosis feature of the lung, stress concentrations were patterned to induce the dilation of tissue openings in the engineered microtissues described in Example 2. In order to further increase local stress levels, rigid micropillars were designed to support the long microtissue. In order to experimentally model the mechanical process of traction bronchiectasis, lung fibroblast-populated long microtissue was induced to fibrosis using TGF-β1 treatment (5 ng/ml). Continuous TGF-β1 treatment for 6 days induced increased expression of α-SMA and pro-collagen type 1 across the entire microtissue, in addition to dilated tissue openings around the micropillars. These effects were absent in untreated conditions. To examine the effects of fenretinide (2 μM and 4 μM doses) in inhibiting traction bronchiectasis, fenretinide was co-administered with TGF-β1 at the beginning of long microtissue culture and remained for 6 days. The unique geometry of the long microtissue allows tissue opening (hole) formation around micropillars under elevated contractile force generated by the differentiated myofibroblasts, mimicking the traction bronchiectasis. As seen in FIG. 8 there are enlarged tissue openings in TGF-β1 treated condition, but tissue openings remained small in both 2 μM and 4 μM fenretinide treated conditions, suggesting these drug concentrations are effective in inhibiting the formation of traction bronchiectasis. FIG. 9 presents the quantified opening area/total tissue area ratio. FIG. 10 demonstrates (A) that the expression level of α-SMA in the long microtissue is strongly inhibited by 4 μM Fenretinide, and (B) a similar trend with the pro-collagen expression. These examples demonstrate that the 4 μM dose of fenretinide was more effective in human pulmonary fibrosis biomarker inhibition, as compared to the 2 μM dose.

### Example 4: Fenretinide Antifibrotic Effects in Intratracheal Bleomycin Mouse Model of Pulmonary Fibrosis (28-Day Model)

Pulmonary fibrosis is a group of complex pathologies of lung, chronic in nature, characterized by injury to the alveolar epithelium, aberrant extracellular matrix (ECM) remodeling and abnormal proliferation of fibroblasts in the pulmonary parenchyma (pulmonary foci). These fibroblasts secrete exaggerated amounts of ECM that subsequently promote aberrant lung architecture remodeling. The bleomycin-induced lung injury in mice is a well-documented pulmonary fibrosis model used to investigate the efficacy of innovative antifibrotic drug entities in reducing pulmonary fibrosis. Brief lung exposure to bleomycin (BLM), a glycopeptide antibiotic, causes epithelial-cell apoptosis, activating an inflammatory wound-healing response that can lead to a temporary excess in deposition of ECM components in the affected tissues. Bleomycin is the most widely used agent, often regarded as the standard in modelling pulmonary fibrosis. The most common route of administration is intratracheal, generally causing an inflammatory response and increased epithelial apoptosis within the first 7 days, followed by 3 days of transitional period, in which inflammation is resolving and fibrosis is detected. The fibrotic stage persists for a period of 3-4 weeks post-BLM and is characterized by excessive deposition of the extracellular matrix, causing areas of fibrosis.

Oral administration of a solid, amorphous dispersion formulation of fenretinide (LAU-7b SDI) was provided as a treatment for reduction of pulmonary fibrosis, in a mouse model of lung fibrosis induced via a single intratracheal administration of bleomycin, for 21 days. The LAU-7b SDI formulation was obtained by spray drying and contains fenretinide in amorphous form in a solid dispersion comprising a matrix polymer, based on the following formula: 40% fenretinide, 59.6% povidone (KOLLIDON® 30), 0.24% butylated hydroxyanisole (BHA) and 0.16% butylated hydroxytoluene (BHT).

Male C57BL/6N mice from Charles River Laboratories, 10-12 weeks of age, weighing around 20-25 g, at the time of their enrolment, received 50 μL of bleomycin sulfate (2.0 mg/kg) by a single intratracheal instillation on Day 1 of the study to induce lung fibrosis. The sham group received an equivalent volume of 0.9% saline. On Day 7 postbleomycin instillation, the animals were randomized in groups of animals based on their body weight loss and enhanced pause airway resistance estimate (PenH value), while the sham group had 6 mice. Oral dosing by gavage (10 mL/kg) with the vehicle (sham and BLM control groups), the test articles (fenretinide 10 mg/kg), and the reference compound (pirfenidone 100 mg/kg) were administered once daily starting from Day 7 to Day 28. Two hours following the last dose on Day 28, all mice were sacrificed, the lungs of each mouse harvested, flushed with 0.9% NaCl and inflated with 10% neutral buffered formalin solution. After embedding and mounting of the lung tissue (transversal slices of 5 μm thickness), the histology slides were stained with hematoxylin/eosin for the Ashcroft score evaluation by a treatment-blinded pathologist and with Sirius red staining for automated fibrosis quantification (Biocelvia, France).

The key endpoints evaluated in this study were Ashcroft score, pulmonary foci and collagen content obtained by histological assessment of the lung tissue at the end of the study. Statistical analysis was made on normally distributed data by one-way ANOVA followed by multiple comparisons (Fisher's LSD post hoc test) to assess the difference between the groups, using Graph Pad Prism Software version 8.0 (San Diego, CA, USA). Differences were considered statistically significant when P values were p<0.05 (*), or p<0.01 (**) or p<0.001 (***).

Ashcroft scale for the evaluation of bleomycin-induced lung fibrosis is the analysis of stained histological samples by visual assessment. The Ashcroft score was assessed in lung tissue collected on Day 28 by treatment-blinded pathologist on H&E stained lung tissue histology slides (3 cross sections/animal) and using the modified Ashcroft scale. Briefly, each lung section was scanned at a magnification of x100 (H&E staining), read by a blinded examiner, and graded from 0 (normal lung) to 8 (total fibrotic obliteration of the field). Criteria for grading lung fibrosis in Ashcroft scale are as follows: Grade 0=normal lung; Grade 1=minimal fibrous thickening of alveolar or bronchiolar walls; Grade 3=moderate thickening of walls without obvious damage to lung architecture; Grade 5=increased fibrosis with definitive damage to lung structure and formation of fibrous bands or small fibrous masses; Grade 7=severe distortion of structure and large fibrous area; Grade 8=total fibrous obliteration of lung fields. The mean value of the grades obtained for each of the three sections was used as the Ashcroft score. Ashcroft score, using the modified scale, is a reliable and reproducible method of visual quantification of bleomycin induced lung fibrosis in H&E stained histological lung sections based on description of the general morphology of alveolar septa and lung structure.

Pulmonary foci (fibrotic tissue) and lung collagen content were quantified in the Sirius red stained lung tissue histology sections by high resolution, automated digital image analysis (Biocellvia, France) on Day 28 after lung fibrosis induction by bleomycin (BLM) instillation and following 21-day oral gavage with fenretinide, pirfenidone (reference drug) or vehicle only. The total sets of lung slices used for the above standard fibrosis evaluation were scanned at ×20 magnification and digital images of entire lung sections were captured using a specialized software. Digital images were then reduced from ×20 to ×2.5 magnification with a pixel size allowing both a high-resolution visualization of morphological structure of pulmonary tissue and a very short processing time for the software analysis (<1 sec per entire lung section). The quantification of the BLM-induced fibrotic alterations has been assessed on the basis of pulmonary tissue density, using Biocellvia proprietary software program allowing the determination of pulmonary tissue density from thousands of micro-tiles (30-56 μm²) crisscrossing the selected pulmonary tissue of entire lung sections. The density of the pulmonary tissue (fibrotic foci and collagen percentage content) was evaluated and their frequency quantified for all individual micro-tiles corresponding to the ratio of the area of the lung tissue inside the micro-tile and the total area of the micro-tile. Full field analysis of 3 lung sections per animal was conducted and the results were expressed as a mean percentage of total surface. This test is recognized as highly reliable, offering reproducible and observer-independent assessment of quantitative analysis of fibrosis.

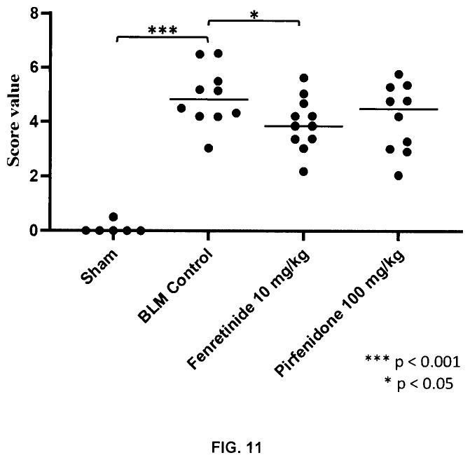

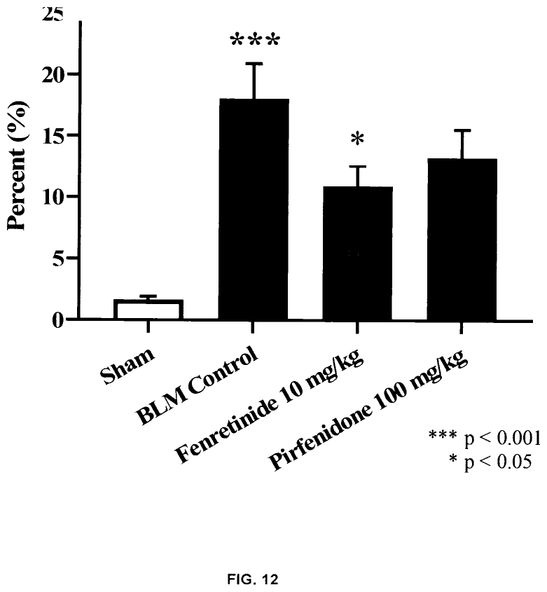

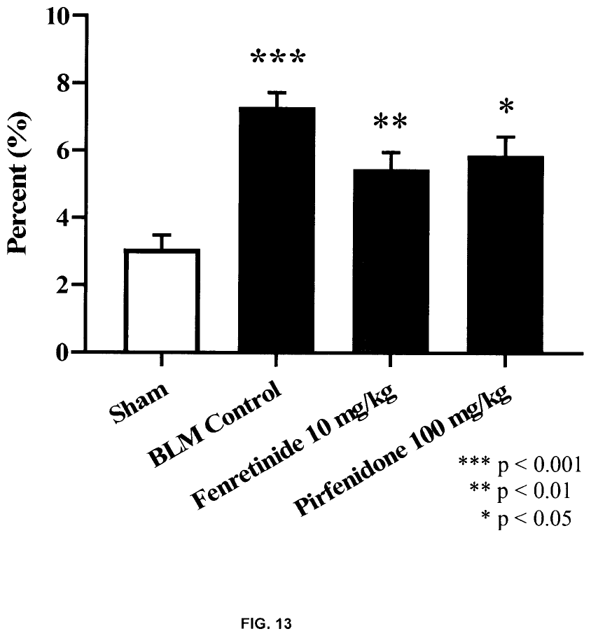

The results of histopathology assessment of the lungs at the end of the study demonstrated the ability of daily oral fenretinide formulation (LAU-7b SDI) treatment to reduce the lung morphological disorganization associated with the fibrosis development in the mouse model of pulmonary fibrosis induced by intratracheal bleomycin instillation. FIG. 11 shows that compared to the sham group (no BLM), the Ashcroft score increased significantly in the group BLM control, receiving only vehicle treatment; and that compared to the BLM control group, fenretinide daily oral dose of 10 mg/kg for 21 consecutive days, reduced significantly (p<0.05) the Ashcroft score, while pirfenidone at the dose of 100 mg/kg (reference drug) did not induce significant improvement. Similarly, FIG. 12 shows the results of the automated histology analysis of the lung tissue showed significant reduction of the percentage of pulmonary foci (fibrotic tissue) in the group receiving 10 mg/kg dose of fenretinide, compared to a nonsignificant effect in the pirfenidone group. Moreover, FIG. 13 demonstrates that the percentage of the collagen content in the lung tissue quantified by automated histology analysis was also significantly reduced by 10 mg/kg fenretinide treatment (p<0.01) and by pirfenidone (p<0.05).

### Example 5: Fenretinide Antifibrotic Effects on Intratracheal Bleomycin Mouse Model of Pulmonary Fibrosis (21-Day Model)

Oral administration of a solid amorphous dispersion formulation of fenretinide (LAU-7b SDI) was evaluated in treating and/or reducing pulmonary fibrosis in mouse model of lung fibrosis induced via a single intratracheal administration of bleomycin followed by vehicle, fenretinide (LAU-7b SDI) or pirfenidone oral treatment for 21 days. The LAU-7b SDI formulation was obtained by spray drying and contains fenretinide in amorphous form in a solid dispersion comprising a matrix polymer, based on the following formula: 40% fenretinide, 59.6% povidone (KOLLIDON® 30), 0.24% butylated hydroxyanisole (BHA) and 0.16% butylated hydroxytoluene (BHT).

Male C57BL/6N mice from Charles River Laboratories, 10-12 weeks of age, weighing around 20-25 g, at the time of their enrolment, received 50 μL of bleomycin sulfate (2.0 mg/kg) by a single intratracheal instillation on Day 1 of the study. The Sham group received an equivalent volume of 0.9% saline. On Day 7 post-bleomycin instillation the animals were randomized in groups of 8 animals based on their body weight loss and Penh value. Oral dosing by gavage (10 mL/kg) with the vehicle (sham and BLM Control groups), LAU-7b SDI (fenretinide at 5 mg/kg and 10 mg/kg dose), and the reference compound (pirfenidone 100 mg/kg) were initiated once daily starting from Day 7 to Day 21. Two hours following the last dose on Day 21 all the mice were sacrificed, the lungs of each mouse were harvested, flushed with 0.9% NaCl and inflated with 10% neutral buffered formalin solution. After embedding and mounting of the lung tissue (transversal slices of 5 μm thickness), the histology slides were stained with hematoxylin/eosin for the Ashcroft score evaluation by a treatment-blinded pathologist, and with Sirius red staining for automated fibrosis quantification (Biocellvia, France).

As in Example 4, the key endpoints evaluated in this study were the Ashcroft score, pulmonary foci and collagen content obtained by histological assessment of the lung tissue at the end of the study. Statistical analysis was made on normally distributed data by one-way ANOVA followed by multiple comparisons (Fisher's LSD post hoc test) to assess the difference between the groups, using Graph Pad Prism Software version 8.0 (San Diego, CA, USA). Differences were considered statistically significant when P values were p<0.05 (*), or p<0.01 (**) or p<0.001 (***). The Ashcroft score was assessed in the lung tissue by treatment-blinded pathologist on the H&E stained histology slides (2 cross sections/animal) and using the modified Ashcroft scale. Briefly, each lung section was scanned at a magnification of ×100 (H&E staining), and graded from 0 (normal lung) to 8 (total fibrotic obliteration of the field). The mean value of the grades obtained for each of the two sections was used as the Ashcroft score.

Pulmonary foci (fibrotic tissue) and lung collagen content were quantified in the Sirius red stained lung tissue histology sections by high resolution, automated digital image analysis (Biocellvia, France) on Day 21 after lung fibrosis induction by bleomycin (BLM) instillation and following a 14-day oral gavage treatment with LAU-7b SDI (fenretinide, two doses), pirfenidone (reference drug) or vehicle only. Full field analysis of 2 lung sections per animal was conducted and the results were expressed as a mean percentage of total surface. This test is considered highly reliable, offering reproducible and observer-independent assessment of quantitative analysis of fibrosis.

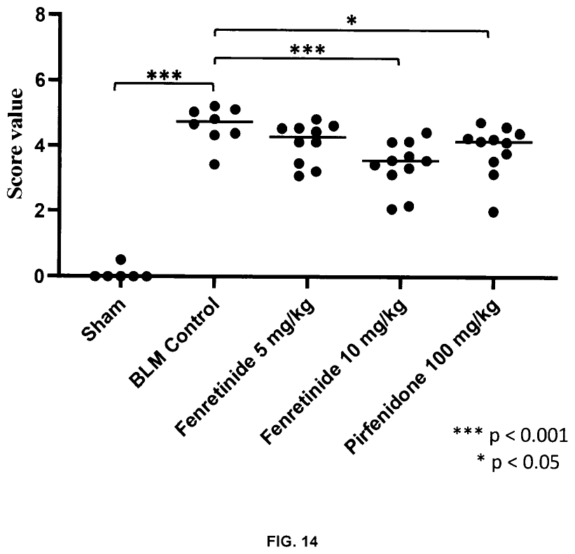

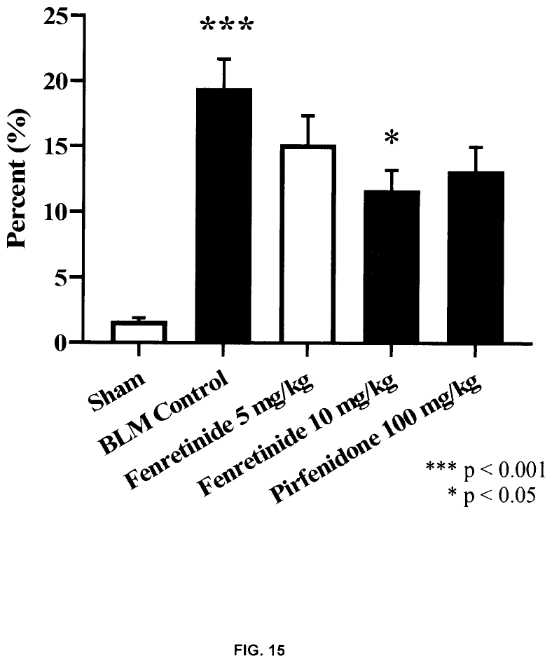

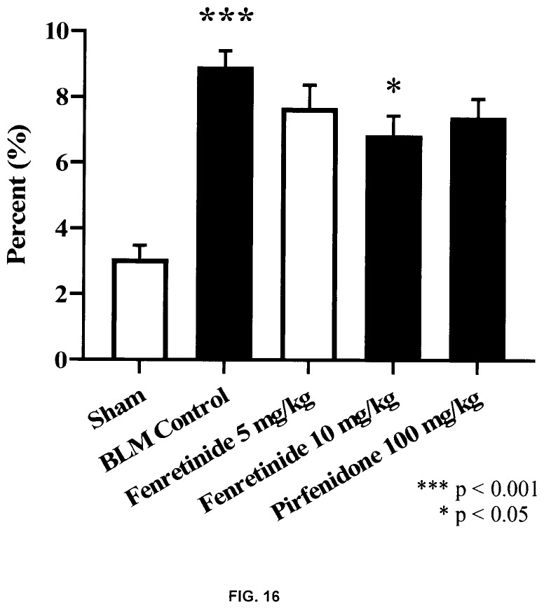

FIG. 14 shows assessment of two histology cuts of the whole left lung, with a significant increase of Ashcroft score (p<0.001), observed in BLM Control animals, receiving daily vehicle treatment, compared to the Sham animals (no BLM) also receiving vehicle treatment only. Compared to the BLM control group both, fenretinide daily oral dose of 10 mg/kg, and pirfenidone at 100 mg/kg for 14 days reduced significantly the Ashcroft score (p<0.001 and p<0.05 respectively). The daily fenretinide oral dose of 5 mg/kg also reduced the Ashcroft score. The automated histological quantification of pulmonary foci and collagen content in the Sirius red stained lung tissue, showed significantly increased percentage of pulmonary foci (percent of total surface) and collagen content in BLM control animals receiving vehicle treatment, vs sham animals (no BLM) also receiving vehicle treatment (p<0.001). Compared to the BLM control group, fenretinide daily oral dose of 10 mg/kg for 14 days, reduced significantly (p<0.05) the percentage of both pulmonary foci (FIG. 15) and collagen content (FIG. 16), while fenretinide dose of 5 mg/kg and pirfenidone at 100 mg/kg did not induce statistically significant reduction.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

1-88 (canceled)

89. A method for treating pulmonary fibrosis, in a human subject in need thereof, comprising administration of a therapeutically effective amount of fenretinide, fenretinide analog or pharmaceutically acceptable salt thereof to such human subject.

90. The method of claim 89, wherein pulmonary fibrosis is associated with one or more interstitial lung diseases (ILD), or with bronchiectasis.

91. The method of claim 90, wherein the one or more interstitial lung diseases is selected from a group consisting of idiopathic pulmonary fibrosis, systemic sclerosis ILD (SSc-ILD) and rheumatoid arthritis ILD (RA-ILD).

92. The method of claim 91, wherein the administration of a therapeutically effective amount of fenretinide, fenretinide analog or pharmaceutically acceptable salt thereof is selected from the group comprising systemic oral administration to a human subject, local administration to the lungs of a human subject by mouth inhalation, and local administration to the lungs of a human subject by nasal inhalation.

93. The method of claim 92, wherein the systemic oral administration to a human subject is by oral administration of 10 mg to 300 mg of fenretinide, fenretinide analog or pharmaceutically acceptable salt thereof which gives rise to a plasma concentration of 1 μM to 3 μM fenretinide or fenretinide analog in said human subject.

94. The method of claim 93, wherein the fenretinide, fenretinide analog or pharmaceutically acceptable salt thereof is present in amorphous solid dispersion.

95. The method of claim 94, wherein the fenretinide is administrated as LAU-7b.

96. The method of claim 92 wherein the local administration to the lungs of a human subject by mouth inhalation, and local administration to the lungs of a human subject by nasal inhalation comprises administration of about 1.8 g/kg to about 3.6 g/kg of fenretinide, fenretinide analog or pharmaceutically acceptable salt to the lungs of such human subject.

97. The method of claim 91, wherein fenretinide, fenretinide analog or pharmaceutically acceptable salt thereof is administered in combination with pirfenidone.

98. The use of fenretinide, fenretinide analog or pharmaceutically acceptable salt thereof in the preparation of a pharmaceutical composition for preventing and/or slowing progression of and/or treating pulmonary fibrosis, in a human subject in need thereof.

99. The pharmaceutical composition of claim 98, wherein pulmonary fibrosis is associated with one or more interstitial lung diseases (ILD), or with bronchiectasis.

100. The pharmaceutical composition of claim 99, wherein the one or more interstitial lung diseases are selected from a group consisting of idiopathic pulmonary fibrosis (IPF), systemic sclerosis ILD (SSc-ILD) and rheumatoid arthritis ILD (RA-ILD).

101. The pharmaceutical composition of claim 100, wherein the administration of a therapeutically effective amount of fenretinide, fenretinide analog or pharmaceutically acceptable salt thereof is selected from the group comprising systemic oral administration to a human subject, local administration to the lungs of a human subject by mouth inhalation, and local administration to the lungs of a human subject by nasal inhalation.

102. The pharmaceutical composition of claim 101, wherein the systemic oral administration to a human subject is by oral administration of 10 mg to 300 mg of fenretinide, fenretinide analog or pharmaceutically acceptable salt thereof which gives rise to a plasma concentration of 1 μM to 3 μM fenretinide or fenretinide analog in said human subject.

103. The pharmaceutical composition of claim 102, wherein the fenretinide, fenretinide analog or salt thereof is present in amorphous solid dispersion.

104. The pharmaceutical composition of claim 103, wherein fenretinide is administered as LAU-7b.

105. The pharmaceutical composition of claim 101 wherein the local administration to the lungs of a human subject by mouth inhalation, and local administration to the lungs of a human subject by nasal inhalation comprises administration of about 1.8 g/kg to about 3.6 g/kg of fenretinide, fenretinide analog or pharmaceutically acceptable salt to the lungs of such human subjects.

106. The pharmaceutical composition of claim 100, wherein fenretinide, fenretinide analog or pharmaceutically acceptable salt thereof is administered in combination with pirfenidone.
